(19) United States
(12) Patent Application Publication
(10) Pub. No.: US 2022/0202857 A1
(43) Pub. Date: Jun. 30, 2022

(54) SYSTEMS AND METHODS FOR REDUCING LOW ATTENUATION PLAQUE AND/OR PLAQUE BURDEN IN PATIENTS

(71) Applicant: HDL Therapeutics, Inc., Vero Beach, FL (US)

(72) Inventors: Hollis Bryan Brewer, JR., Potomac, MD (US); Michael M. Matin, Short Hills, NJ (US)

(21) Appl. No.: 17/571,225

(22) Filed: Jan. 7, 2022

Related U.S. Application Data
(63) Continuation-in-part of application No. 17/315,509, filed on May 10, 2021.
Application No. 17/315,509 is a continuation of application No. 17/021,883, filed on Sep. 15, 2020, now Pat. No. 11,033,582.
Application No. 17/021,883 is a continuation of application No. 16/225,210, filed on Dec. 19, 2018, now Pat. No. 10,821,133.
Application No. 16/225,210 is a continuation-in-part of application No. 17/012,410, filed on Sep. 4, 2020.
Application No. 17/012,410 is a continuation of application No. 16/198,672, filed on Nov. 21, 2018, now Pat. No. 11,027,052.
Application No. 16/198,672 is a continuation-in-part of application No. 17/012,396, filed on Sep. 4, 2020.
Application No. 17/012,396 is a continuation of application No. 16/198,672, filed on Nov. 21, 2018, now Pat. No. 11,027,052.
Application No. 16/198,672 is a continuation-in-part of application No. 16/698,193, filed on Nov. 27, 2019.
Application No. 16/698,193 is a continuation-in-part of application No. 16/409,543, filed on May 10, 2019.
Application No. 16/409,543 is a continuation-in-part of application No. 15/909,765, filed on Mar. 1, 2018.
Application No. 15/909,765 is a continuation-in-part of application No. 15/876,808, filed on Jan. 22, 2018.
Application No. 16/698,193 is a continuation-in-part of application No. 16/225,210, filed on Dec. 19, 2018, now Pat. No. 10,821,133.
Application No. 16/698,193 is a continuation-in-part of application No. 16/198,672, filed on Nov. 21, 2018, now Pat. No. 11,027,052.
Application No. 16/698,193 is a continuation-in-part of application No. 16/046,830, filed on Jul. 26, 2018.
Application No. 16/046,830 is a continuation-in-part of application No. 15/909,765, filed on Mar. 1, 2018.
Application No. 15/909,765 is a continuation-in-part of application No. 16/409,543, filed on May 10, 2019.
Application No. 16/409,543 is a continuation-in-part of application No. 15/909,765, filed on Mar. 1, 2018.
Application No. 15/909,765 is a continuation-in-part of application No. 15/876,808, filed on Jan. 22, 2018.
Application No. 15/876,808 is a continuation-in-part of application No. 16/046,830, filed on Jul. 26, 2018.
Application No. 16/046,830 is a continuation-in-part of application No. 15/909,765, filed on Mar. 1, 2018.
Application No. 15/909,765 is a continuation-in-part of application No. 15/876,808, filed on Jan. 22, 2018.
Application No. 15/876,808 is a continuation-in-part of application No. 15/909,765, filed on Mar. 1, 2018.
Application No. 15/909,765 is a continuation-in-part of application No. 15/876,808, filed on Jan. 22, 2018.
Application No. 15/876,808 is a continuation-in-part of application No. 15/876,808, filed on Jan. 22, 2018.
(60) Provisional application No. 63/296,928, filed on Jan. 6, 2022.
(60) Provisional application No. 63/262,423, filed on Oct. 12, 2021.
(60) Provisional application No. 63/135,001, filed on Jan. 8, 2021.
(60) Provisional application No. 62/611,098, filed on Dec. 28, 2017.
(60) Provisional application No. 62/589,919, filed on Nov. 22, 2017.
(60) Provisional application No. 62/589,919, filed on Nov. 22, 2017.
(60) Provisional application No. 62/773,388, filed on Nov. 30, 2018.
(60) Provisional application No. 62/700,804, filed on Jul. 19, 2018.
(60) Provisional application No. 62/670,615, filed on May 11, 2018.
(60) Provisional application No. 62/516,100, filed on Jun. 6, 2017.
(60) Provisional application No. 62/465,262, filed on Mar. 1, 2017.
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.
(60) Provisional application No. 62/611,098, filed on Dec. 28, 2017.
(60) Provisional application No. 62/589,919, filed on Nov. 22, 2017.
(60) Provisional application No. 62/537,581, filed on Jul. 27, 2017.
(60) Provisional application No. 62/700,804, filed on Jul. 19, 2018.
(60) Provisional application No. 62/670,615, filed on May 11, 2018.
(60) Provisional application No. 62/537,581, filed on Jul. 27, 2017.
(60) Provisional application No. 62/516,100, filed on Jun. 6, 2017.
(60) Provisional application No. 62/465,262, filed on Mar. 1, 2017.
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.
(60) Provisional application No. 62/537,581, filed on Jul. 27, 2017.
(60) Provisional application No. 62/537,581, filed on Jul. 27, 2017.
(60) Provisional application No. 62/516,100, filed on Jun. 6, 2017.
(60) Provisional application No. 62/465,262, filed on Mar. 1, 2017.
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.
(60) Provisional application No. 62/537,581, filed on Jul. 27, 2017.
(60) Provisional application No. 62/516,100, filed on Jun. 6, 2017.
(60) Provisional application No. 62/465,262, filed on Mar. 1, 2017.
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.
(60) Provisional application No. 62/516,100, filed on Jun. 6, 2017.
(60) Provisional application No. 62/516,100, filed on Jun. 6, 2017.
(60) Provisional application No. 62/449,416, filed on Jan. 23, 2017.

(51) Int. Cl. A61K 35/16; A61K 38/17

(52) U.S. Cl. CPC A61K 35/16; A61K 38/17

(57) ABSTRACT

The present specification is directed to systems, apparatus and methods for delaying, halting and stabilizing, reversing, or improving the progression of at least one lipid-related condition. The progression of, stabilizing, or improving the at least one lipid-related condition is treated by determining if plaque is present in at least one artery of the patient and determining a type of plaque present in at least one artery of the patient. If a fibrous plaque is present, determining a treatment protocol based on Fractional Flow Reserve. If a low attenuation plaque is present, determining a treatment protocol based on plaque burden. Depending on the extent of plaque, the treatment protocol includes administering to the patient a high density lipoprotein composition derived from mixing a blood fraction with a lipid removing agent.

### CROSS-REFERENCE

The present application relies on, for priority, U.S. Patent Provisional Application No. 63/296,928, titled “System and Method for Reducing Low Attenuation Plaque and/or Plaque Burden in Patients”, filed on Jan. 6, 2022.

The present application also relies on, for priority, U.S. Patent Provisional Application No. 63/262,423, titled “Methods for Prophylactically Preventing, Slowing the Progression of, or Treating Cerebral Amyloid Angiopathy, Alzheimer's Disease and/or Acute Stroke”, filed on Oct. 12, 2021.

The present application also relies on, for priority, U.S. Patent Provisional Application No. 63/135,001, titled “Methods for Treating Lipid-Related Dysfunction in Lupus Patients”, filed on Jan. 8, 2021.

The present application is also a continuation-in-part application of U.S. patent application Ser. No. 17/315,509, titled “Methods for Preserving and Administering Pre-Beta High Density Lipoprotein Having a Predetermined Minimum Level of Degradation” and filed on May 10, 2021, which is a continuation application of U.S. patent application Ser. No. 17/021,883, of the same title, filed on Sep. 15, 2020, and issued as U.S. Pat. No. 11,033,582 on Sep. 15, 2020, which is a continuation application of U.S. patent application Ser. No. 16/225,210, titled “Methods for Preserving and Administering Pre-Beta High Density Lipoprotein Extracted from Human Plasma”, filed on Dec. 19, 2018, and issued as U.S. Pat. No. 10,821,133 on Nov. 3, 2020, which, in turn, relies on, for priority, U.S. Provisional Patent Application No. 62/611,098, titled “Methods for Treating Cholesterol-Related Diseases” and filed on Dec. 28, 2017.

The present application is also a continuation-in-part application of U.S. patent application Ser. No. 17/012,410, titled “Systems for Removing Air from the Fluid Circuits of a Plasma Processing System” and filed on Sep. 4, 2020, which is a continuation application of U.S. patent application Ser. No. 16/198,672, titled “Systems and Methods for Priming Fluid Circuits of a Plasma Processing System”, filed on Nov. 21, 2018, and issued as U.S. Pat. No. 11,027,052 on Jun. 8, 2021, which, in turn, relies on U.S. Provisional Patent Application No. 62/589,919, entitled “Systems and Methods for Causing Regression of Arterial Plaque” and filed on Nov. 22, 2017, for priority.

The present application is also a continuation-in-part application of U.S. patent application Ser. No. 17/012,396, titled “Systems for Removing Air from the Fluid Circuits of a Plasma Processing System” and filed on Sep. 4, 2020, which is a continuation application of U.S. patent application Ser. No. 16/198,672, titled “Systems and Methods for Priming Fluid Circuits of a Plasma Processing System”, filed on Nov. 21, 2018, and issued as U.S. Pat. No. 11,027,052 on Jun. 8, 2021, which, in turn, relies on U.S. Provisional Patent Application No. 62/589,919, entitled “Systems and Methods for Causing Regression of Arterial Plaque” and filed on Nov. 22, 2017, for priority.

The present application is also a continuation-in-part application of U.S. patent application Ser. No. 16/698,193, titled “Methods for Treating Lipid-Related Diseases Including Xanthomas, Carotid Artery Stenoses, and Cerebral Atherosclerosis” and filed on Nov. 27, 2019, which relies on U.S. Patent Provisional Application No. 62/773,388, titled “Methods for Treating Cholesterol Related Diseases” and filed on Nov. 30, 2018, for priority.

U.S. patent application Ser. No. 16/698,193 is also a continuation-in-part application of U.S. patent application Ser. No. 16/409,543, titled “Methods for Prophylactically Preventing, Slowing the Progression of, or Treating Cerebral Amyloid Angiopathy, Alzheimer's Disease and/or Acute Stroke” and filed on May 10, 2019, which relies on U.S. Provisional Patent Application No. 62/700,804, titled “Methods for Prophylactically Preventing, Slowing the Progression of, or Treating Cerebral Amyloid Angiopathy, Alzheimer's Disease and/or Acute Stroke” and filed on Jul. 19, 2018 and U.S. Provisional Patent Application No. 62/670,615, of the same title and filed on May 11, 2018, for priority.

U.S. patent application Ser. No. 16/409,543 is also a continuation-in-part application of U.S. patent application Ser. No. 15/909,765, titled “Methods for Prophylactically Preventing, Slowing the Progression of, or Treating Alzheimer's Disease” and filed on Mar. 1, 2018, which relies on U.S. Provisional Patent Application No. 62/537,581, titled “Methods for Treating Cholesterol-Related Diseases” and filed on Jul. 27, 2017, U.S. Provisional Patent Application No. 62/516,100, entitled “Methods for Treating Cholesterol-Related Diseases” and filed on Jun. 6, 2017, and U.S. Provisional Patent Application No. 62/465,262, entitled “Method for Treating Familial Hypercholesterolemia” and filed on Mar. 1, 2017, for priority.

U.S. patent application Ser. No. 15/909,765 is also a continuation-in-part application of U.S. patent application Ser. No. 15/876,808, titled “Methods for Treating Cholesterol-Related Diseases” and filed on Jan. 22, 2018, which, in turn, relies on U.S. Provisional Patent Application No. 62/516,100, titled “Methods for Treating Cholesterol-Related Diseases” and filed on Jun. 6, 2017, U.S. Provisional Patent Application No. 62/465,262, titled “Method for Treating Familial Hypercholesterolemia” and filed on Mar. 1, 2017, and U.S. Provisional Patent Application No. 62/449,416, titled “Method for Treating Familial Hypercholesterolemia” and filed on Jan. 23, 2017, for priority.

U.S. patent application Ser. No. 16/698,193 is also a continuation-in-part application of U.S. patent application Ser. No. 16/225,210, entitled “Methods for Preserving and Administering Pre-Beta High Density Lipoprotein Extracted from Human Plasma”, filed on Dec. 19, 2018, and issued as U.S. Pat. No. 10,821,133 on Nov. 3, 2020, which relies on U.S. Provisional Patent Application No. 62/611,098, titled “Methods for Treating Cholesterol-Related Diseases” and filed on Dec. 28, 2017, for priority.

U.S. patent application Ser. No. 16/698,193 is also a continuation-in-part application of U.S. patent application Ser. No. 16/198,672, titled “Systems and Methods for Priming Fluid Circuits of a Plasma Processing System”, filed on Nov. 21, 2018, and issued as U.S. Pat. No. 11,027,052 on Jun. 8, 2021, which relies on U.S. Provisional Patent Application No. 62/589,919, titled “Systems and Methods for Causing Regression of Arterial Plaque” and filed on Nov. 22, 2017, for priority.

U.S. patent application Ser. No. 16/698,193 is also a continuation-in-part application of U.S. patent application Ser. No. 16/046,830, titled “Methods for Treating Cholesterol-Related Diseases Using Administered Solutions Having Increased Pre-Beta HDL Particles” and filed on Jul. 26, 2018, which relies on U.S. Provisional Patent Application No. 62/537,581, titled “Method for Treating Cholesterol-Related Diseases” and filed on Jul. 27, 2017, for priority.

U.S. patent application Ser. No. 16/046,830 is also a continuation-in-part application of U.S. patent application Ser. No. 15/909,765, entitled “Methods for Prophylactically Preventing, Slowing the Progression of, or Treating Alzheimer's Disease”, and filed on Mar. 1, 2018, which, in turn, relies on U.S. Provisional Patent Application No. 62/537,581, entitled “Method for Treating Cholesterol-Related Diseases” and filed on Jul. 27, 2017, U.S. Provisional Patent Application No. 62/516,100, entitled “Methods for Treating Cholesterol-Related Diseases” and filed on Jun. 6, 2017, and U.S. Provisional Patent Application No. 62/465,262, entitled “Method for Treating Familial Hypercholesterolemia” and filed on Mar. 1, 2017, for priority.

The present application is also a continuation-in-part application of U.S. patent application Ser. No. 16/409,543, titled “Methods for Prophylactically Preventing, Slowing the Progression of, or Treating Cerebral Amyloid Angiopathy, Alzheimer's Disease and/or Acute Stroke” and filed on May 10, 2019, which relies on U.S. Provisional Patent Application No. 62/700,804, titled “Methods for Prophylactically Preventing, Slowing the Progression of, or Treating Cerebral Amyloid Angiopathy, Alzheimer's Disease and/or Acute Stroke” and filed on Jul. 19, 2018 and U.S. Provisional Patent Application No. 62/670,615, of the same title and filed on May 11, 2018, for priority.

The present application is also a continuation-in-part application of U.S. patent application Ser. No. 16/046,830, tilted “Methods for Treating Cholesterol-Related Diseases Using Administered Solutions Having Increased Pre-Beta HDL Particles” and filed on Jul. 26, 2018, which relies on U.S. Provisional Patent Application No. 62/537,581, entitled “Method for Treating Cholesterol-Related Diseases” and filed on Jul. 27, 2017, for priority.

The present application is also a continuation-in-part application of U.S. patent application Ser. No. 15/909,765, entitled “Methods for Prophylactically Preventing, Slowing the Progression of, or Treating Alzheimer's Disease” and filed on Mar. 1, 2018, which relies on U.S. Provisional Patent Application No. 62/465,262, entitled “Method for Treating Familial Hypercholesterolemia” and filed on Mar. 1, 2017, U.S. Provisional Patent Application No. 62/516,100, entitled “Methods for Treating Cholesterol-Related Diseases” and filed on June 6, and U.S. Provisional Patent Application No. 62/537,581, entitled “Methods for Treating Cholesterol-Related Diseases” and filed on Jul. 27, 2017, for priority.

The present application is also a continuation-in-part application of U.S. patent application Ser. No. 15/876,808, titled “Methods for Treating Cholesterol-Related Diseases”, and filed on Jan. 22, 2018, which, in turn, relies on U.S. Provisional Patent Application No. 62/516,100, entitled “Methods for Treating Cholesterol-Related Diseases” and filed on Jun. 6, 2017, U.S. Provisional Patent Application No. 62/465,262, entitled “Method for Treating Familial Hypercholesterolemia” and filed on Mar. 1, 2017, and U.S. Provisional Patent Application No. 62/449,416, entitled “Method for Treating Familial Hypercholesterolemia” and filed on Jan. 23, 2017, for priority.

The above-mentioned applications are herein incorporated by reference in their entirety.

### FIELD

The method of the present specification provides for a treatment procedure in which lipid from HDL is selectively removed to create a modified HDL particle, while leaving LDL particles substantially intact, and the modified HDL particle is administered to an individual in order to delay, halt and stabilize, reverse or improve the progression of a disease or pathophysiologic process associated with a lipid-related, low attenuation plaque-related or increased plaque burden-related condition.

### BACKGROUND

Cholesterol is synthesized by the liver or obtained from dietary sources. LDL is responsible for transferring cholesterol from the liver to tissues at different sites in the body. However, if LDL collects on the arterial walls, it undergoes oxidation caused by oxygen free radicals liberated from the body's chemical processes and interacts deleteriously with the blood vessels. The modified LDL causes white blood cells in the immune system to gather at the arterial walls, forming a fatty substance called plaque (which may be fibrous plaque, lipid-filled plaque, or low attenuation plaque and/or low attenuation non-calcified plaque) and injuring cellular layers that line blood vessels. The modified oxidized LDL also reduces the level of nitric oxide, which is responsible for relaxing the blood vessels and thereby allowing the blood to flow freely. As this process continues, the arterial walls slowly constrict, resulting in hardening of the arteries and thereby reducing blood flow. The gradual build-up of plaque can result in blockage of a blood vessel that supplies blood to an organ such as, for example, the heart, kidneys or brain. The plaque build-up can also occur in peripheral vessels such as the legs and this condition is known as peripheral arterial disease (“PAD”).

In contrast to LDL, high plasma HDL levels are desirable because they play a major role in “reverse cholesterol transport”, where the excess cholesterol is transferred from tissue sites to the liver where it is eliminated. Optimal total cholesterol levels are 200 mg/dl or below with a LDL cholesterol level of 160 mg/dl or below and a HDL-cholesterol level of 45 mg/dl for men and 50 mg/dl for women. Lower LDL levels are recommended for individuals with a history of elevated cholesterol, atherosclerosis, or coronary artery disease. High levels of LDL increase the lipid content in coronary arteries resulting in formation of plaques, which may include low attenuation or lipid-filled plaques that are vulnerable to rupture. HDL has been shown to decrease the lipid content in the low-attenuation or lipid-filled plaques, reducing the probability of rupture. In the last several years, clinical trials of low density lipoprotein (LDL)-lowering drugs have definitively established that reductions in LDL are associated with a 30-45% decrease in clinical cardiovascular disease (CVD) events. CVD events include events occurring in diseases such as HoFH, HeFH, and peripheral arterial disease (PAD). Despite lowered LDL, however, many patients continue to have cardiac events. Low levels of HDL are often present in high-risk subjects with CVD, and epidemiological studies have identified HDL as an independent risk factor that modulates CVD risk. In addition to epidemiologic studies, other evidence suggests that raising HDL would reduce the risk of CVD. There has been increasing interest in changing plasma HDL levels by dietary, pharmacological, or genetic manipulations as a potential strategy for the treatment of CVD including HoFH, HeFH, Ischemic stroke, CAD, ACS, and peripheral arterial disease (PAD), which are described below.

The protein component of LDL, known as apolipoprotein-B (ApoB), and its products, comprise atherogenic elements. Elevated plasma LDL levels and reduced HDL levels are recognized as primary causes of coronary disease. ApoB is in highest concentration in LDL particles and is not present in HDL particles. Apolipoprotein A-I (ApoA-I) and apolipoprotein A-II (ApoA-II) are found in HDL. Other apolipoproteins, such as ApoC and its subtypes (C-I, C-II and C-III), ApoD, and ApoE are also found in HDL. ApoC and ApoE are also observed in LDL particles.

Numerous major classes of HDL particles including HDL2b, HDL2a, HDL3a, HDL3b and HDL3 have been reported. Various forms of HDL particles have been described on the basis of electrophoretic mobility on agarose as two major populations, a major fraction with α-HDL mobility and a minor fraction with migration similar to VLDL. This latter fraction has been called pre-β HDL and these particles are the most efficient HDL particle subclass for inducing cellular cholesterol efflux.

The HDL lipoprotein particles are comprised of ApoA-I, phospholipids, and cholesterol. The pre-β HDL particles are considered to be the first acceptors of cellular free cholesterol and are essential in eventually transferring free and esterified cholesterol to α-HDL. Pre-β HDL particles may transfer cholesterol to α-HDL or be converted to α-HDL. The alpha HDL transfers cholesterol to the liver, where excess cholesterol can be removed from the body.

HDL levels are inversely correlated with atherosclerosis and coronary artery disease. Once cholesterol-carrying α-HDL reaches the liver, the α-HDL particles divest of the cholesterol and transfer the cholesterol to the liver. The α-HDL particles (divested of cholesterol) are subsequently converted to pre-β HDL particles and exit the liver, which then serve to pick up additional cholesterol within the body and are converted back to α-HDL, thus repeating the cycle.

It has been shown that hypercholesterolemia and inflammation are two dominant mechanisms implicated in the development of atherosclerosis. A common cardiovascular disease that occurs due to development of atherosclerosis (hardening and narrowing of the arteries) within the elastic lining inside a coronary artery, is Coronary Artery Disease (CAD), also known as Ischemic Heart Disease (IHD). On the basis of a statistical data collected from 2009 to 2012, an estimated 15.5 million Americans ≥20 years of age have CAD. The total CAD prevalence in the United States is 6.2% of adults ≥20 years of age.

An accurate decrease in blood flow in the coronary arteries may result in part of the heart muscle unable to function properly. This condition is known as Acute Coronary Syndrome (ACS). A conservative estimate for the number of hospital discharges with ACS in the United States in 2010 is 625,000.

While conventional diagnostic methods for risk of cardiovascular events include cardiovascular risk scores and the presence of obstructive stenoses in the coronary vasculature, coronary computed tomography angiography (CCTA or coronary CTA) is a more recent technique that has the ability to visualize both the coronary artery lumen and any surrounding atherosclerotic plaque, such as in lumen walls. Further coronary CTA has the ability to identify and differentiate lesions, which are defined in terms of calcifications (large or speckled), presence of low-attenuation plaques, positive remodeling, and presence of napkin ring sign (NRS). Thus, both coronary plaque burden and plaque type may be assessed. The quantification of the burden of calcified, noncalcified, and low attenuation plaque using CCTA may be used as a diagnostic tool to drive treatment and improve therapeutic outcomes. With CCTA, the presence of a low attenuation plaque is characterized by a lipid rich necrotic core. Low-attenuation plaques in both the coronary and carotid arteries are inherently unstable as they are fatty, necrotic, and have a high risk of rupturing. Low attenuation plaques may be present in both the lumen and embedded in the lumen wall of the arteries, which makes them particularly dangerous.

By way of example, lupus patients have a significantly higher burden of low attenuation non-calcified plaque (LANCP) compared to their healthy counterparts. To date, most treatments, however, are aimed at suppressing or alleviating the symptoms of lupus, rather than addressing the associated lipid-related conditions, such as the buildup of low attenuation non-calcified plaque. To date, most treatments, however, are aimed at suppressing or alleviating the symptoms of lupus, rather than addressing the associated lipid-related conditions, such as the buildup of low attenuation non-calcified plaque.

Similarly, renal arterial stenosis (RAS) refers to a blockage in an artery that supplies blood to the kidney and is characterized in two forms: a) smooth muscle or fibrous plaque or b) cholesterol-filled plaque (lipid-filled or low attenuation plaque). This condition, generally known as renal arterial stenosis, decreases blood flow to the kidney and can result in high blood pressure.

There is a need, therefore, for a comprehensive treatment protocol that may delay, halt and stabilize, reverse or improve the progression of diseases or pathophysiologic processes related to the presence of low attenuation plaque in patients. Accordingly, what is needed is a method for reducing low attenuation plaque as quantified by a reduction in overall plaque burden.

### SUMMARY

The following embodiments and aspects thereof are described and illustrated in conjunction with systems, tools and methods, which are meant to be exemplary and illustrative, and not limiting in scope. The present specification discloses numerous embodiments.

In some embodiments, the present specification describes a method for delaying a progression of, stabilizing, or improving at least one lipid-related condition in a patient, comprising: determining a type of plaque present in at least one artery of the patient, wherein said plaque may be found within the lumen of the at least one artery, wherein said type of plaque is at least one of a fibrous plaque or a low attenuation plaque; if, based on said determining, the plaque is a low attenuation plaque, subsequently determining a volume of the low attenuation plaque; calculating a plaque burden using the volume of low attenuation plaque; administering a delipidation treatment protocol for the patient if the plaque burden is greater than or equal to a predetermined threshold value, wherein the delipidation treatment protocol comprises acquiring a blood fraction from the patient, mixing said blood fraction with a lipid removing agent to yield a delipidated blood fraction, deriving a high-density lipoprotein composition from the delipidated blood fraction, and administering to the patient the high-density lipoprotein composition.

Optionally, the at least one lipid-related condition is lupus and wherein the low attenuation plaque is determined based on monitoring of a pathophysiological change indicative of lupus. Still optionally, after administering to the patient the high-density lipoprotein composition, a percentage of obstruction of the patient's physiological parameters indicative of lupus stabilizes and does not experience a further increase in obstruction.

Optionally, the at least one lipid-related condition is atheroembolic renal disease, and wherein the at least one artery supplies blood to the patient's kidney. Optionally, after administering to the patient the high-density lipoprotein composition, the patient experiences a reduction of plasma burden in the at least one artery that supplies blood to the patient's kidney.

Optionally, the at least one lipid-related condition is a cardiac condition, and wherein the at least one artery supplies blood to the patient's heart.

Optionally, the high-density lipoprotein composition derived from mixing the blood fraction with the lipid removing agent is delivered to the patient via infusion therapy in a dosage ranging from 1 mg/kg to 250 mg/kg.

Optionally, the high-density lipoprotein composition derived from mixing the blood fraction of the patient with the lipid removing agent is delivered to the patient via infusion therapy at a rate of 999 mL per hour plus or minus 100 mL per hour.

Optionally, after administering to the patient the high-density lipoprotein composition, the patient experiences a regression of the accumulation of low attenuation plaque in the at least one artery.

Optionally, after administering to the patient the high-density lipoprotein composition, the patient experiences a reduction of plasma burden in the at least one artery. Still optionally, if the reduction in plasma burden is below 4%, the treatment protocol is halted. Still optionally, if the plasma burden is reduced, but still greater than or equal to 4%, the treatment protocol is resumed or modified.

Optionally, the method further includes connecting the patient to a device for withdrawing blood; withdrawing blood from the patient; and separating blood cells from the blood to yield the blood fraction containing high density lipoproteins and low density lipoproteins.

Optionally, the predetermined threshold value for the plaque burden is 4%.

Optionally, if, based on said determining, the plaque is the fibrous plaque, the method includes performing a fractional flow reserve assessment on the patient. Optionally, the method further comprises determining a percent of blockage due to lipid content.

Optionally, if the fractional flow reserve of the patient is equal to or above 80% and the percent of blockage due to lipid content is in a range of 20% to 30%, the delipidation treatment protocol is administered to the patient. Optionally, if the fractional flow reserve of the patient is below 80% or the percent of blockage due to lipid content is below 20% or above 30%, the delipidation treatment protocol is not administered to the patient.

Optionally, if the plaque burden is less than 4%, the delipidation treatment protocol is not administered to the patient.

Optionally, the at least one lipid-related condition is homozygous familial hypercholesterolemia, heterozygous familial hypercholesterolemia, Ischemic stroke, coronary artery disease, acute coronary syndrome, or peripheral arterial disease (PAD).

Optionally, the method further includes determining, if the plaque is a fibrous plaque, a fractional flow reserve; determining a third treatment protocol if the fractional flow reserve is less than or equal to 79%; and determining a fourth treatment protocol if the fractional flow reserve is greater than 80%. Still optionally, the third treatment protocol comprises embedding a stent in the patient, and wherein the fourth treatment protocol comprises administering a medical therapy other than embedding a stent in the patient or administering the delipidation protocol to the patient.

In some embodiments, the present specification discloses a method for delaying a progression of, stabilizing, or improving at least one lipid-related condition in a patient, comprising: determining if plaque is present in at least one artery of the patient, wherein said plaque may be found within the lumen or lumen wall of the at least one artery, wherein said plaque is one of a fibrous plaque or a low attenuation plaque; determining, if the plaque is a low attenuation plaque, a volume of the low attenuation plaque; calculating a plaque burden using the volume of low attenuation plaque; administering a treatment protocol for the patient if the plaque burden is greater than or equal to a predetermined threshold, wherein the treatment protocol comprises administering to the patient a high density lipoprotein composition derived from mixing a blood fraction with a lipid removing agent.

Optionally, the at least one lipid-related condition is lupus, wherein the low attenuation plaque is determined based on monitoring of a pathophysiological change indicative of lupus.

Optionally, the at least one lipid-related condition is atheroembolic renal disease, and wherein the at least one artery supplies blood to the patient's kidney.

Optionally, the at least one lipid-related condition is a cardiac condition, and wherein the at least one artery supplies blood to the patient's heart.

Optionally, after administering to the patient the high density lipoprotein composition, a percentage of obstruction of the patient's physiological parameters indicative of lupus stabilizes and does not experience a further increase in obstruction.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour+/−100 mL/hr.

Optionally, after administering to the patient the high density lipoprotein composition, the patient experiences a regression of the accumulation of low attenuation plaque in the at least one artery.

Optionally, after administering to the patient the high density lipoprotein composition, the patient experiences a reduction of plasma burden in the at least one artery.

Optionally, the high density lipoprotein composition is derived by obtaining the blood fraction from the patient, wherein the blood fraction has high-density lipoproteins; mixing the blood fraction with the lipid removing agent to yield modified high-density lipoproteins; separating the modified high-density lipoproteins; and delivering the modified high-density lipoproteins to the patient.

Optionally, the method further comprises connecting the patient to a device for withdrawing blood; withdrawing blood from the patient; and separating blood cells from the blood to yield the blood fraction containing high density lipoproteins and low density lipoproteins.

Optionally, the modified high density lipoproteins have an increased concentration of pre-beta high density lipoproteins relative to the high density lipoproteins from the blood fraction prior to mixing.

Optionally, the modified high density lipoproteins have a concentration of alpha high density lipoproteins in addition to pre-beta high density lipoproteins from the blood fraction prior to mixing.

Optionally, the high density lipoprotein composition is derived by obtaining the blood fraction from an individual other than the patient, wherein the blood fraction has high-density lipoproteins; mixing the blood fraction with the lipid removing agent to yield modified high-density lipoproteins; separating the modified high-density lipoproteins; and delivering the modified high-density lipoproteins to the patient.

In some embodiments, the present specification is directed towards a method for delaying the progression of, stabilizing, or improving at least one lipid-related condition in a patient, comprising: determining if plaque is present in at least one artery of the patient; calculating a plaque burden in the at least one artery; based on a determination of the plaque burden in the at least one artery of the patient, determining a treatment protocol for the patient; and, administering to the patient a high density lipoprotein composition derived from mixing a blood fraction, having unmodified high density lipoproteins, with a lipid removing agent to yield modified high density lipoproteins, wherein the modified high density lipoproteins have an increased concentration of pre-beta high density lipoprotein relative to the unmodified high density lipoproteins, if the plaque burden is greater than or equal to a predetermined minimum threshold percentage.

Optionally, the predetermined minimum threshold percentage for the plaque burden is 4%.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour+/−100 mL/hr.

Optionally, after administering to the patient the high density lipoprotein composition, the patient experiences a regression of the accumulation of low attenuation plaque in the at least one artery.

In some embodiments, the present specification discloses a method for reducing accumulated low attenuation plaque in a patient, comprising: determining if plaque is present in at least one artery of the patient, wherein said plaque may be found within the lumen or lumen wall of the at least one artery; determining a level of plaque burden in said at least one artery; and, based on the level of the plaque burden, determining a treatment protocol for the patient, wherein the treatment protocol comprises administering to the patient a high density lipoprotein composition derived from mixing a blood fraction with a lipid removing agent.

Optionally, the accumulated low attenuation plaque is reduced by 10 to 50%.

Optionally, the treatment protocol is administered if the level of plaque burden is greater than or equal to 4%.

In some embodiments, the present specification discloses a method for delaying a progression of, stabilizing, or improving at least one lipid-related condition in a patient, comprising: determining if plaque is present in at least one artery of the patient, wherein said plaque may be found within the lumen or lumen wall of the at least one artery, and wherein said plaque is one of a fibrous plaque or a low attenuation plaque; determining, if the plaque is a low attenuation plaque, a volume of the low attenuation plaque; calculating a plaque burden using the volume of low attenuation plaque; determining a first treatment protocol for the patient if the plaque burden is greater than or equal to a predetermined minimum threshold, wherein the first treatment protocol comprises administering to the patient a high density lipoprotein composition derived from mixing a blood fraction with a lipid removing agent; and determining a second treatment protocol for the patient if the plaque burden is less than the predetermined threshold, wherein the second treatment protocol comprises administering a medical therapy.

Optionally, the predetermined minimum threshold is 4%.

Optionally, the at least one lipid-related condition is homozygous familial hypercholesterolemia, heterozygous familial hypercholesterolemia, Ischemic stroke, coronary artery disease, acute coronary syndrome, or peripheral arterial disease, and wherein the at least one artery supplies blood to the patient's heart.

Optionally, the at least one lipid-related condition is atheroembolic renal disease, and wherein the at least one artery supplies blood to the patient's kidney.

Optionally, the at least one lipid-related condition is lupus.

Optionally, the method further comprises determining, if the plaque is a fibrous plaque, a fractional flow reserve; determining a third treatment protocol if the fractional flow reserve is less than or equal to 79%; and determining a fourth treatment protocol if the fractional flow reserve is greater than 80%.

Optionally, the third treatment protocol corresponds to embedding a stent in the patient, and wherein the fourth treatment protocol comprises administering a medical therapy.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour+/−100 mL/hr.

Optionally, the high density lipoprotein composition is derived by obtaining the blood fraction from the patient, wherein the blood fraction has high-density lipoproteins; mixing the blood fraction with the lipid removing agent to yield modified high-density lipoproteins; separating the modified high-density lipoproteins; and delivering the modified high-density lipoproteins to the patient.

Optionally, the method further comprises connecting the patient to a device for withdrawing blood; withdrawing blood from the patient; and separating blood cells from the blood to yield the blood fraction containing high density lipoproteins and low density lipoproteins.

Optionally, the modified high density lipoproteins have an increased concentration of pre-beta high density lipoproteins relative to the high density lipoproteins from the blood fraction prior to mixing.

Optionally, the modified high density lipoproteins have a concentration of alpha high density lipoproteins in addition to pre-beta high density lipoproteins from the blood fraction prior to mixing.

Optionally, the method further comprises deriving the high density lipoprotein composition by obtaining the blood fraction from an individual other than the patient, wherein the blood fraction has high-density lipoproteins; mixing the blood fraction with the lipid removing agent to yield modified high-density lipoproteins; separating the modified high-density lipoproteins; and delivering the modified high-density lipoproteins to the patient.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 is a schematic representation of a plurality of components used in accordance with some embodiments of the present specification to achieve the processes disclosed herein;

FIG. 2 is a pictorial illustration of an exemplary embodiment of a configuration of a plurality of components used in accordance with some embodiments of the present specification to achieve the processes disclosed herein;

FIG. 3 shows a decision tree illustrating a protocol for administering modified HDL particles to a patient, in accordance with some embodiments of the present specification.

FIG. 4 is a table illustrating the types of treatments that may be provided for different compositions of plaque, in accordance with some embodiments of the present specification;

FIG. 5 is a flowchart describing plurality of exemplary steps of a therapeutic protocol for treating a lupus patient, in accordance with an embodiment of the present specification;

FIG. 6 is a flow chart delineating the steps of treating cardiovascular diseases using treatment systems and methods in accordance with embodiments of the present specification; and

FIG. 7 is another flow chart delineating the steps of treating lipid-related renal diseases, such as Atheroembolic Renal Disease (AERD), using the treatment systems and methods in accordance with embodiments of the present specification.

### DETAILED DESCRIPTION

The present specification relates to methods and systems for treating low attenuation plaque, cholesterol, or lipid-related conditions. Embodiments of the present specification treat the condition through systems, apparatuses and methods useful for removing lipid from α-High Density Lipoprotein (α-HDL) particles derived primarily from plasma of the patient thereby creating modified HDL particles with reduced lipid content, particularly reduced cholesterol content. Embodiments of the present specification create these modified HDL particles with reduced lipid content without substantially modifying LDL particles. Embodiments of the present specification modify original α-HDL particles to yield modified HDL particles that have an increased concentration of pre-β HDL relative to the original HDL. The newly formed derivatives of HDL particles (modified HDL) are administered to the patient to enhance cellular cholesterol efflux and treat cholesterol or lipid-related conditions, including, but not limited to delaying, halting and stabilizing, reversing or improving the progression of diseases or pathophysiologic processes related to the presence of low attenuation plaque, which may include necrotic and degenerative material, in one or more arteries in patients.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

The term “fluid” may be defined as fluids from animals or humans that contain lipids or lipid containing particles, fluids from culturing tissues and cells that contain lipids and fluids mixed with lipid-containing cells. For purposes of this invention, decreasing the amount of lipids in fluids includes decreasing lipids in plasma and particles contained in plasma, including but not limited to HDL particles. Fluids include, but are not limited to: biological fluids; such as blood, plasma, serum, lymphatic fluid, cerebrospinal fluid, peritoneal fluid, pleural fluid, pericardial fluid, various fluids of the reproductive system including, but not limited to, semen, ejaculatory fluids, follicular fluid and amniotic fluid; cell culture reagents such as normal sera, fetal calf serum or serum derived from any animal or human; and immunological reagents, such as various preparations of antibodies and cytokines from culturing tissues and cells, fluids mixed with lipid-containing cells, and fluids containing lipid-containing organisms, such as a saline solution containing lipid-containing organisms. A preferred fluid treated with the methods of the present invention is plasma.

The term “lipid” may be defined as any one or more of a group of fats or fat-like substances occurring in humans or animals. The fats or fat-like substances are characterized by their insolubility in water and solubility in organic solvents. The term “lipid” is known to those of ordinary skill in the art and includes, but is not limited to, complex lipid, simple lipid, triglycerides, fatty acids, glycerophospholipids (phospholipids), true fats such as esters of fatty acids, glycerol, cerebrosides, waxes, and sterols such as cholesterol and ergosterol.

The term “extraction solvent” or “lipid removing agent” may be defined as one or more solvents used for extracting lipids from a fluid or from particles within the fluid. This solvent enters the fluid and remains in the fluid until removed by other subsystems. Suitable extraction solvents include solvents that extract or dissolve lipid, including but not limited to phenols, hydrocarbons, amines, ethers, esters, alcohols, halohydrocarbons, halocarbons, and combinations thereof. Examples of suitable extraction solvents are ethers, esters, alcohols, halohydrocarbons, or halocarbons which include, but are not limited to di-isopropyl ether (DIPE), which is also referred to as isopropyl ether, diethyl ether (DEE), which is also referred to as ethyl ether, lower order alcohols such as butanol, especially n-butanol, ethyl acetate, dichloromethane, chloroform, isoflurane, sevoflurane (1,1,1,3,3,3-hexafluoro-2-(fluoromethoxy) propane-d3), perfluorocyclohexanes, trifluoroethane, cyclofluorohexanol, and combinations thereof.

The term “patient” refers to animals and humans, which may be either a fluid source to be treated with the methods of the present invention or a recipient of derivatives of HDL particles and or plasma with reduced lipid content.

The term “HDL particles” encompasses several types of particles defined based on a variety of methods such as those that measure charge, density, size and immuno-affinity, including but not limited to electrophoretic mobility, ultracentrifugation, immunoreactivity and other methods known to one of ordinary skill in the art. Such HDL particles include but are not limited to the following: α-HDL, pre-β HDL (including pre-β1 HDL, pre-β2 HDL and pre-β3HDL), HDL2 (including HDL2a and HDL2b), HDL3, VHDL, LpA-I, LpA-II, LpA-I/LpA-II (for a review see Barrans et al., Biochemica Biophysica Acta 1300; 73-85,1996). Accordingly, practice of the methods of the present invention creates modified HDL particles. These modified derivatives of HDL particles may be modified in numerous ways including but not limited to changes in one or more of the following metabolic and/or physico-chemical properties (for a review see Barrans et al., Biochemica Biophysica Acta 1300; 73-85, 1996); molecular mass (kDa); charge; diameter; shape; density; hydration density; flotation characteristics; content of cholesterol; content of free cholesterol; content of esterified cholesterol; molar ratio of free cholesterol to phospholipids; immuno-affinity; content, activity or helicity of one or more of the following enzymes or proteins: ApoA-I, ApoA-II, ApoD, ApoE, ApoJ, ApoA-IV, cholesterol ester transfer protein (CETP), lecithin; cholesterol acyltransferase (LCAT); capacity and/or rate for cholesterol binding, capacity and/or rate for cholesterol transport.

“Coronary computed tomography angiography” (referred to as “CCTA” or coronary CTA) refers to a diagnostic technique that has the ability to visualize both the coronary artery lumen and any surrounding atherosclerotic plaque, such as in lumen walls. Using CCTA, a clinician is able to assess both plaque type (fibrous/fibromuscular plaque, low attenuation plaque or any other type) and coronary plaque burden.

In the case of fibrous or fibromuscular plaque, “fractional flow reserve” or “FFR” is used to refer to a measurement of pressure differences across a coronary artery stenosis (a narrowing, usually due to atherosclerosis) to determine the likelihood that the stenosis impedes oxygen delivery to the heart muscle. Fractional flow reserve is defined as the pressure after (distal to) a stenosis relative to the pressure before the stenosis and is presented as an absolute number. An FFR value of 0.70 means that a given stenosis causes a 30% drop in blood pressure. Thus, FFR is used to express the maximal flow down a vessel in the presence of stenosis compared to the maximal flow in the hypothetical absence of stenosis. A decrease in blood flow, which is measured in terms of blood pressure using FFR, results in a decrease in oxygen delivery via blood (blood oxygen delivery).

The term “low attenuation plaque” refers to plaque that, as measured using CCTA (coronary computed tomography angiography) have a radiodensity of less than 30 Hounsfield units. In embodiments, the minimum plaque size that may be measured by CCTA is defined as having a volume larger than 3 mm³ (that is, a cross-sectional area of about 3 mm² and a length of about 1 mm). It should be noted that the term “low attenuation non-calcified plaque” may be used throughout this specification as well, but should not be construed as limiting. By way of definition, coronary plaque calcification is a later manifestation of atherosclerosis while earlier stages are represented by noncalcified or mixed composition plaques containing extracellular lipid and fibrous tissue. The devices, methods, and treatments as described in the present specification may be applied to any low attenuation plaque.

The term “plaque burden” is defined as the relevant plaque volume (in mm³) divided by the vessel volume of the region under investigation, multiplied by 100 to arrive at a percentage and is expressed as the percentage. The plaque burden is typically summed on a per patient basis. The plaque volume may be measured for total plaque, calcified plaque, non-calcified plaque, and low attenuation plaque, wherein the plaque burden is calculated for each.

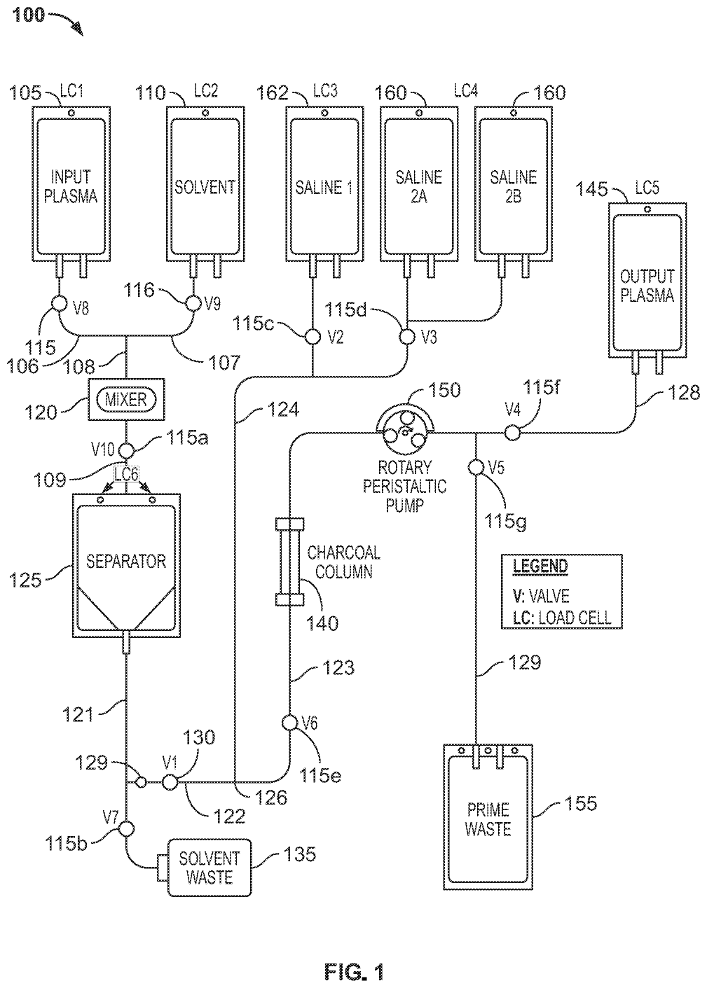

FIG. 1 illustrates an exemplary embodiment of a system and its components used to achieve the delipidation methods of the present specification. The figure depicts an exemplary basic component flow diagram defining elements of the HDL modification system 100. Embodiments of the components of system 100 are utilized after obtaining a blood fraction from a patient or another individual (donor). Plasma, separated from the blood, is brought in a sterile bag to system 100 for further processing. The plasma may be separated from blood using a known plasmapheresis device. The plasma may be collected from the patient into a sterile bag using standard apheresis techniques. The plasma is then brought in the form of a fluid input to system 100 for further processing. In embodiments, system 100 is not connected to the patient at any time and is a discrete, stand-along system for delipidating plasma. The patient's plasma is processed by system 100 and brought back to the patient's location to be reinfused back into the patient. In alternate embodiments, the system may be a continuous flow system that is connected to the patient in which both plasmapheresis and delipidation are performed in an extracorporeal, parallel system and the delipidated plasma product is returned to the patient.

A fluid input 105 (containing blood plasma) is provided and connected via fluid line or tubing 106 to a mixing device 120. A solvent input 110 is provided and also connected via fluid line or tubing 107 to mixing device 120. In embodiments, valve 115 is positioned within fluid line 106 and is used to control the flow of fluid from fluid input 105. In embodiments, valve 116 is positioned within fluid line 107 and is used to control the flow of fluid from solvent input 110. It should be appreciated that the fluid input 105 contains any fluid that includes HDL particles, including plasma having LDL particles or devoid of LDL particles, as discussed above. It should further be appreciated that solvent input 110 can include a single solvent, a mixture of solvents, or a plurality of different solvents that are mixed at the point of solvent input 110. While depicted as a single solvent container, solvent input 110 can comprise a plurality of separate solvent containers. Embodiments of types of solvents that may be used are discussed above.

Mixer 120 mixes fluid from fluid input 105 and solvent from solvent input 110, via fluid line 108, to yield a fluid-solvent mixture. In embodiments, mixer 120 is capable of using a shaker bag mixing method with the input fluid and input solvent in a plurality of batches, such as 1, 2, 3 or more batches. An exemplary mixer is a Barnstead Labline orbital shaker table. In alternative embodiments, other known methods of mixing are utilized. Once formed, the fluid-solvent mixture is directed, through tubing or fluid line 109 and controlled by at least one valve 115a, to a separator 125. In an embodiment, separator 125 is capable of performing bulk solvent separation through gravity separation in a funnel-shaped bag.

In separator 125, the fluid-solvent mixture separates into a first layer and second layer. The first layer comprises a mixture of solvent and lipid that has been removed from the HDL particles. The first layer is transported through fluid line 121, and directed to valve 115b, positioned within fluid line 121, to a first waste container 135. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 125, the second layer is transported through fluid line 122 to a solvent extraction device 140. In an embodiment, a pressure sensor 129 and valve 130 is positioned in fluid line 122, in the flow stream to control the flow of the second layer to solvent extraction device 140. Valve 115e is positioned in fluid flow line 123 to also control the flow of fluid toward the solvent extraction device.

The opening and closing of valves 115, 116 to enable the flow of fluid from input containers 105, 110 may be timed using mass balance calculations derived from weight determinations of the fluid inputs 105, 110 and separator 125. For example, the valve 115b between separator 125 and first waste container 135 and valve 130 between separator 125 and solvent extraction device 140 open after the input masses (fluid and solvent) substantially balances with the mass in separator 125 and a sufficient period of time has elapsed to permit separation between the first and second layers. Depending on what solvent is used, and therefore which layer settles to the bottom of separator 125, either valve 115b between separator 125 and first waste container 135 is opened or valve 130 between separator 125 and solvent extraction device 140 is opened. One of ordinary skill in the art would appreciate that the timing of the opening is dependent upon how much fluid is in the first and second layers and would further appreciate that it is preferred to keep valve 115b between separator 125 and first waste container 135 open just long enough to remove all of the first layer and some of the second layer, thereby ensuring that as much solvent as possible has been removed from the fluid being sent to solvent extraction device 140.

In embodiments, an infusion grade fluid (“IGF”) may be employed via one or more inputs 160, through fluid line 124 and via valve 115d, which is in fluid communication with the fluid flow line 122 (which is also in fluid communication with fluid line 121) at junction 126 leading from separator 125 to solvent extraction device 140 for priming. In an embodiment, saline is employed as the infusion grade priming fluid in at least one of inputs 160. In an embodiment, 0.9% sodium chloride (saline) is employed. In other embodiments, glucose may be employed as the infusion grade priming fluid in any one of inputs 160.

In embodiments, a glucose input 162 (via valve 115c and leading to fluid path 124) and one or more saline inputs 160 (via valve 115d and leading to fluid path 124) are in fluid communication with the fluid flow line 122, which is in communication with fluid flow line 121 and fluid flow line 123, creating a fluid flow path from separator 125 to solvent extraction device 140. Thus, valves 115c and 115d are positioned in the flow stream from glucose input 162 and saline input 160 respectively, to fluid flow line 124 and directed toward fluid flow line 122, which is in fluid communication with fluid flow line 121 and fluid flow line 123, which forms the flow path from separator 125 to solvent extraction device 140.

IGF such as saline and/or glucose are incorporated into embodiments of the present specification in order to prime solvent extraction device 140 prior to operation of the system. In embodiments, saline is used to prime most of the fluid communication lines and solvent extraction device 140. If priming is not required, the IGF inputs are not employed. Where such priming is not required, the glucose and saline inputs are not required. Also, one of ordinary skill in the art would appreciate that the glucose and saline inputs can be replaced with other primers if required by the solvent extraction device 140.

In some embodiments, solvent extraction device 140 is a charcoal column designed to remove the specific solvent used in solvent input 110. An exemplary solvent extraction device 140 is an Asahi Hemosorber charcoal column, or the Bazter/Gambro Adsorba 300C charcoal column or any other charcoal column that is employed in blood hemoglobin perfusion procedures. A pump 150 is used to move the second layer from separator 125, through solvent extraction device 140, and to an output container 145, through fluid flow path 128, within which an optional valve 115f is positioned between pump 150 and output container 145. In embodiments, pump 150 is a rotary peristaltic pump, such as a Masterflex Model 77201-62.

The first layer is directed to a first waste container 135 that is in fluid communication with separator 125 through tubing and at least one valve 115b. Additionally, other waste, if generated, can be directed from the fluid path from solvent extraction device 140 to a second waste container 155, through fluid flow line 129. Optionally, in an embodiment, a valve 115g is included within fluid flow line 129, in the path from the solvent extraction device 140 to the second waste container 155. In an embodiment, first waste container 135 is used for solvent waste while second waste container 155 is used for prime waste.

In an embodiment of the present specification, gravity is used, wherever practical, to move fluid through each of the plurality of components. For example, gravity is used to drain input plasma 105 and input solvent 110 into mixer 120. Where mixer 120 comprises a shaker bag and separator 125 comprises a funnel bag, fluid is moved from the shaker bag to the funnel bag and, subsequently, to first waste container 135, if appropriate, using gravity.

In an additional embodiment, not shown in FIG. 1, the output fluid in output container 145 is subjected to a solvent detection system, or lipid removing agent detection system, to determine if any solvent, or other undesirable component, is in the output fluid. In embodiments, a solvent sensor is only employed in a continuous flow system. In one embodiment, the output fluid is subjected to sensors that are capable of determining the concentrations of solvents introduced in the solvent input, such as n-butanol or di-isopropyl ether. The output fluid is returned to the bloodstream of the patient and the solvent concentrations must be below a predetermined level to carry out this operation safely. In embodiments, the sensors are capable of providing such concentration information on a real-time basis and without having to physically transport a sample of the output fluid, or air in the headspace, to a remote device. The resultant separated modified HDL particles are then introduced to the bloodstream of the patient.

In one embodiment, molecularly imprinted polymer technology is used to enable surface acoustic wave sensors. A surface acoustic wave sensor receives an input, through some interaction of its surface with the surrounding environment, and yields an electrical response, generated by the piezoelectric properties of the sensor substrate. To enable the interaction, molecularly imprinted polymer technology is used. Molecularly imprinted polymers are plastics programmed to recognize target molecules, like pharmaceuticals, toxins or environmental pollutants, in complex biological samples. The molecular imprinting technology is enabled by the polymerization of one or more functional monomers with an excess of a crosslinking monomer in presence of a target template molecule exhibiting a structure similar to the target molecule that is to be recognized, i.e. the target solvent.

The use of molecularly imprinted polymer technology to enable surface acoustic wave sensors can be made more specific to the concentrations of targeted solvents and are capable of differentiating such targeted solvents from other possible interferents. As a result, the presence of acceptable interferents that may have similar structures and/or properties to the targeted solvents would not prevent the sensor from accurately reporting existing respective solvent concentrations.

Alternatively, if the input solvent comprises certain solvents, such as n-butanol, electrochemical oxidation could be used to measure the solvent concentration. Electrochemical measurements have several advantages. They are simple, sensitive, fast, and have a wide dynamic range. The instrumentation is simple and not affected by humidity. In one embodiment, the target solvent, such as n-butanol, is oxidized on a platinum electrode using cyclic voltammetry. This technique is based on varying the applied potential at a working electrode in both the forward and reverse directions, at a predefined scan rate, while monitoring the current. One full cycle, a partial cycle, or a series of cycles can be performed. While platinum is the preferred electrode material, other electrodes, such as gold, silver, iridium, or graphite, could be used. Although, cyclic voltammetric techniques are used, other pulse techniques such as differential pulse voltammetry or square wave voltammetry may increase the speed and sensitivity of measurements.

Embodiments of the present specification expressly cover any and all forms of automatically sampling and measuring, detecting, and analyzing an output fluid, or the headspace above the output fluid. For example, such automated detection can be achieved by integrating a mini-gas chromatography (GC) measuring device that automatically samples air in the output container, transmits it to a GC device optimized for the specific solvents used in the delipidation process, and, using known GC techniques, analyzes the sample for the presence of the solvents.

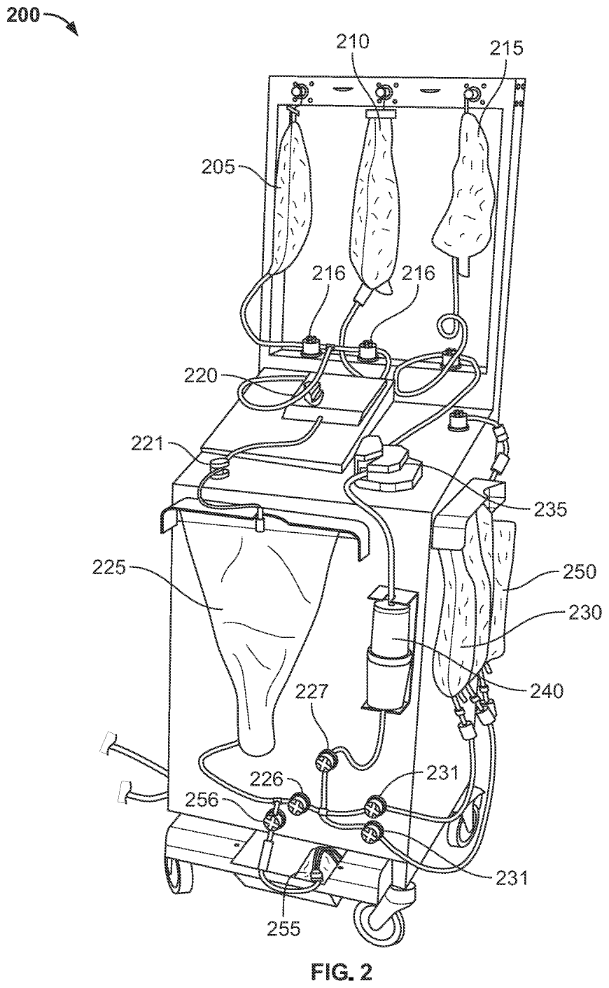

Referring back to FIG. 2, suitable materials for use in any of the apparatus components as described herein include materials that are biocompatible, approved for medical applications that involve contact with internal body fluids, and in compliance with U.S. PVI or ISO 10993 standards. Further, the materials do not substantially degrade from, for instance, exposure to the solvents used in the present specification, during at least a single use. The materials are sterilizable either by radiation or ethylene oxide (EtO) sterilization. Such suitable materials are capable of being formed into objects using conventional processes, such as, but not limited to, extrusion, injection molding and others. Materials meeting these requirements include, but are not limited to, nylon, polypropylene, polycarbonate, acrylic, polysulfone, polyvinylidene fluoride (PVDF), fluoroelastomers such as VITON, available from DuPont Dow Elastomers L.L.C., thermoplastic elastomers such as SANTOPRENE, available from Monsanto, polyurethane, polyvinyl chloride (PVC), polytetrafluoroethylene (PTFE), polyphenylene ether (PFE), perfluoroalkoxy copolymer (PFA), which is available as TEFLON PFA from E.I. du Pont de Nemours and Company, and combinations thereof.

Valves 115, 115a, 115b, 115c, 115d, 115e, 115f, 115g, 116 and any other valve used in each embodiment may be composed of, but are not limited to, pinch, globe, ball, gate or other conventional valves. In some embodiments, the valves are occlusion valves such as Acro Associates' Model 955 valve. However, the present specification is not limited to a valve having a particular style. Further, the components of each system described in accordance with embodiments of the present specification may be physically coupled together or coupled together using conduits that may be composed of flexible or rigid pipe, tubing or other such devices known to those of ordinary skill in the art.

FIG. 2 illustrates an exemplary configuration of a system used in accordance with some embodiments of the present specification to achieve the processes disclosed herein. Referring to FIG. 2, a configuration of basic components of the HDL modification system 200 is shown. A fluid input 205 is provided and connected via tubing to a mixing device 220. A solvent input 210 is provided and also connected via tubing to a mixing device 220. Preferably valves 216 are used to control the flow of fluid from fluid input 205 and solvent from solvent input 210. It should be appreciated that the fluid input 205 preferably contains any fluid that includes HDL particles, including plasma having LDL particles or devoid of LDL particles, as discussed above. It should further be appreciated that solvent input 210 can include a single solvent, a mixture of solvents, or a plurality of different solvents that are mixed at the point of solvent input 210. While depicted as a single solvent container, solvent input 210 can comprise a plurality of separate solvent containers. The types of solvents that are used and preferred are discussed above.

The mixer 220 mixes fluid from fluid input 205 and solvent from solvent input 210 to yield a fluid-solvent mixture. Preferably, mixer 220 is capable of using a shaker bag mixing method with the input fluid and input solvent in a plurality of batches, such as 1, 2, 3 or more batches. Once formed, the fluid-solvent mixture is directed, through tubing and controlled by at least one valve 221, to a separator 225. In a preferred embodiment, separator 225 is capable of performing bulk solvent separation through gravity separation in a funnel-shaped bag.

In the separator 225, the fluid-solvent mixture separates into a first layer and second layer. The first layer comprises a mixture of solvent and lipid that has been removed from the HDL particles. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 225, the second layer is transported through tubing to a solvent extraction device 240. Preferably, a pressure sensor 226 and valve 227 is positioned in the flow stream to control the flow of the second layer to the solvent extraction device 240.

Preferably, a glucose input 230 and saline input 250 is in fluid communication with the fluid path leading from the separator 225 to the solvent extraction device 240. A plurality of valves 231 is also preferably incorporated in the flow stream from the glucose input 230 and saline input 250 to the tubing providing the flow path from the separator 225 to the solvent extraction device 240. Glucose and saline are incorporated into the present specification in order to prime the solvent extraction device 240 prior to operation of the system. Where such priming is not required, the glucose and saline inputs are not required. Also, one of ordinary skill in the art would appreciate that the glucose and saline inputs can be replaced with other primers if the solvent extraction device 240 requires it.

The solvent extraction device 240 is preferably a charcoal column designed to remove the specific solvent used in the solvent input 210. An exemplary solvent extraction device 240 is an Asahi Hemosorber charcoal column. A pump 235 is used to move the second layer from the separator 225, through the solvent extraction device 240, and to an output container 215. The pump is preferably a peristaltic pump, such as a Masterflex Model 77201-62.

The first layer is directed to a waste container 255 that is in fluid communication with separator 225 through tubing and at least one valve 256. Additionally, other waste, if generated, can be directed from the fluid path connecting solvent extraction device 240 and output container 215 to waste container 255.

Preferably, an embodiment of the present specification uses gravity, wherever practical, to move fluid through each of the plurality of components. For example, preferably gravity is used to drain the input plasma 205 and input solvent 210 into the mixer 220. Where the mixer 220 comprises a shaker bag and separator 225 comprises a funnel bag, fluid is moved from the shaker bag to the funnel bag and, subsequently, to the waste container 255, if appropriate, using gravity.

In general, the present specification preferably comprises configurations wherein all inputs, such as input plasma and input solvents, disposable elements, such as mixing bags, separator bags, waste bags, solvent extraction devices, and solvent detection devices, and output containers are in easily accessible positions and can be readily removed and replaced by a technician.

To enable the operation of the above-described embodiments of the present specification, it is preferable to supply a user of such embodiments with a packaged set of components, in kit form, comprising each component required to practice embodiments of the present specification. The kit may include an input fluid container (i.e. a high density lipoprotein source container), a lipid removing agent source container (i.e. a solvent container), disposable components of a mixer, such as a bag or other container, disposable components of a separator, such as a bag or other container, disposable components of a solvent extraction device (i.e. a charcoal column), an output container, disposable components of a waste container, such as a bag or other container, solvent detection devices, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container.

In one embodiment, a kit comprises a plastic container having disposable components of a mixer, such as a bag or other container, disposable components of a separator, such as a bag or other container, disposable components of a waste container, such as a bag or other container, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container. Disposable components of a solvent extraction device (i.e. a charcoal column), the input fluid, the input solvent, and solvent extraction devices may be provided separately.

### Therapeutic Protocol Guidelines for Administering Modified HDL Particles

In accordance with aspects of the present specification, treated plasma containing modified HDL particles with reduced lipid and/or increased pre-β concentration is administered to a patient in accordance with a plurality of therapeutic protocols.

In some embodiments, therapy is based on a level of severity of the lipid-related condition or cardiac disease state, as measured by the volume of low attenuation plaque. In various embodiments, the therapeutic protocols may only be administered if the amount or volume of low attenuation plaque is measured at a minimum threshold amount. Stated differently, therapy will not be provided if low attenuation plaque is below X amount, regardless of total plaque or total cholesterol levels (which, based on historical approaches, would have previously warranted therapy).

In some embodiments, therapy may be based on a quantitative assessment of atherosclerotic plaque, using coronary computer tomography angiography (CCTA), which may be expressed as a plaque burden. The plaque volume may be measured for total plaque, calcified plaque, non-calcified plaque, and low attenuation plaque, wherein the plaque burden is calculated for each. The term “plaque burden” is defined as the relevant plaque volume (in mm³) divided by the vessel volume of the region under investigation, multiplied by 100 to arrive at a percentage and is expressed as the percentage. The plaque burden is typically summed on a per patient basis. Thus, in embodiments, plaque burden may be measured as calcified plaque burden, noncalcified plaque burden, low-attenuation plaque burden, and/or total coronary plaque burden. In some embodiments, a patient is eligible for treatment with the modified HDL particle-based therapy of the present specification, if the patient presents with a plaque burden greater than or equal to a predefined target or minimum threshold of 4%.

In various embodiments, the following guidelines are provided for determining if a patient is eligible for therapy based on a minimum low attenuation plaque volume, which may also be expressed as a minimum plaque burden.
The provision of therapy is not initiated based on any patient's pre-specified range of the level of background LDL-C, but rather, the presence of low attenuation plaque.
In all indications, as described below, a minimum low attenuation plaque is defined as a quantity of plaque that current CT angiography instruments can detect and measure. For example, a documented greater than or equal to one (1) low attenuation plaque in the left main artery, left anterior descending artery, left circumflex artery, and/or right coronary artery with a minimum plaque volume of 3 mm³ in the target lesion on baseline CCTA can be considered a minimum low attenuation plaque.
Inclusion criteria may also include a patient presenting with a known established coronary artery disease based on a past medical history of at least one of the following: documented myocardial infarction, coronary artery bypass graft (CABG) surgery, percutaneous coronary intervention (PCI), invasive coronary angiography, coronary computed tomography angiography (CCTA), and/or elevated coronary calcium score, defined as >100 Agatson units by computed tomography.

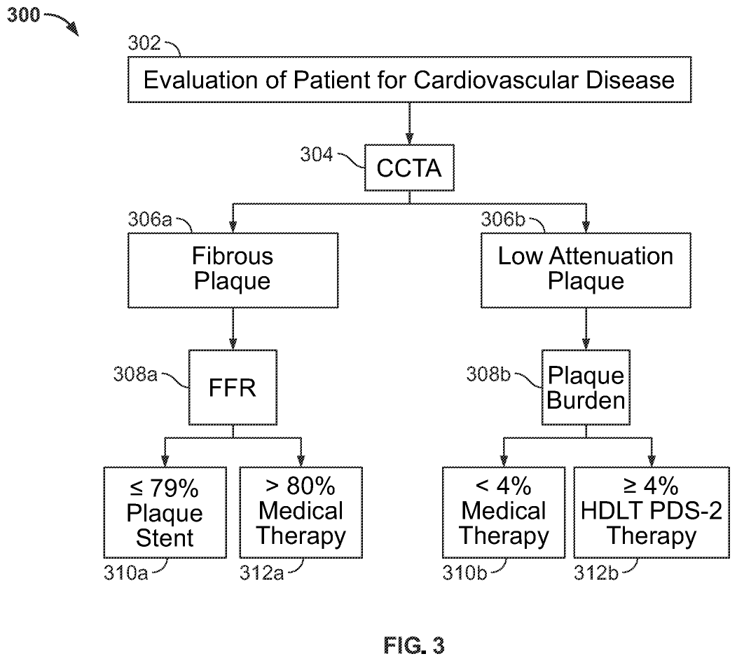

FIG. 3 illustrates a decision tree 300 describing a protocol for administering modified HDL particles to a patient, in accordance with some embodiments of the present specification. Referring now to FIG. 3, decision point 302 is indicative of a scenario where a patient is evaluated for cardiovascular disease. At decision point 304, the patient undergoes a CCTA (coronary computed tomography angiography) to determine the presence of obstructive stenoses and/or plaque in the coronary vasculature. As known the persons of ordinary skill in the art, CCTA has the ability to visualize both the coronary artery lumen and any surrounding atherosclerotic plaque. This allows for an assessment or determination of both plaque type (fibrous plaque or low attenuation plaque) and plaque burden (relevant plasma volume divided by vessel volume times 100).

Based on the CCTA, the identified plaque type is assessed as being fibrous plaque at decision point 306a or low attenuation plaque at decision point 306b. For fibrous plaque, FFR (Fractional Flow Reserve) is used as a parameter at decision point 308a to determine a type of treatment/intervention. If the FFR is less than or equal to 79% an invasive treatment is recommended, at decision point 310a, where a stent is embedded through physical intervention. If the FFR is greater than 80% an appropriate medical therapy is recommended at decision point 312a. That is, the patient may be treated with medications such as, for example, statins.

On the other hand, for low attenuation plaque, plaque burden is used as a parameter at decision point 308b to determine a type of treatment/intervention. If the plaque burden is less than a predefined target/threshold of 4% an appropriate medical therapy is recommended at decision point 310b. However, if the plaque burden is greater than or equal to the predefined target/threshold of 4% the patient is administered modified HDL particle-based therapy of the present specification, at decision point 312b. Several indications and protocols are discussed below.

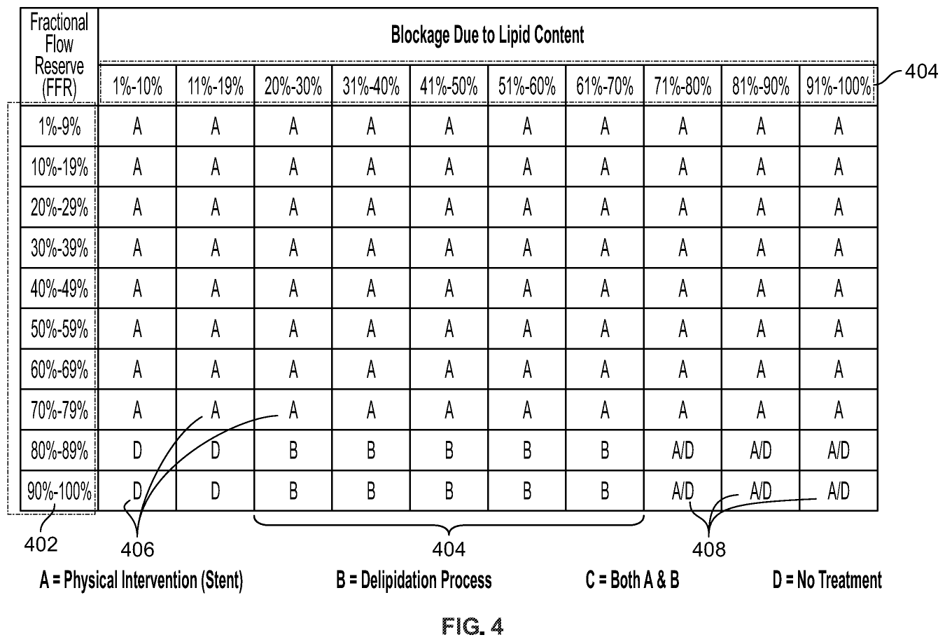

FIG. 4 is a table illustrating the types of treatments that may be provided for different compositions of plaque (including both fibrous/fibromuscular and low attenuation plaque) determined from the diagnosis and treatment protocol of FIG. 3, in accordance with some embodiments of the present specification. The table compares different types of treatments that may be administered for combinations of various ranges of a Fractional Flow Reserve (FFR) 402, which is indicative of a rate of flow of blood after a blockage (which, in turn, is indicative of blood oxygen delivery), provided in terms of percentage (or fraction) of Fractional Flow Reserve, and various ranges of physical blockage due to lipid content 404, provided in terms of percentage of blockage due to lipid content. Referring to the table, each cell, such as cells 406, corresponds to a combination of a range 402 (indicative of FFR) and a range 404 (indicative of the percentage or extent of blockage due to lipid content), which further indicates at least one method of treatment that may be suitable for that combination.

In embodiments, the different types of treatments are coded as A, B, C, and D. Treatment type ‘A’ corresponds to an invasive treatment process where a stent is embedded through physical intervention (for example, steps 306a, 308a and 310a of FIG. 3 are illustrative of a decision flow leading to treatment type ‘A’). Treatment type ‘B’ corresponds to implementing the treatment methods of selectively modifying HDL particles, in accordance with the embodiments of the present specification (for example, steps 306b, 308b and 312b are illustrative of a decision flow leading to treatment type ‘B’). In some embodiments, if a patient presents with additional, remaining lesions that are classified as low attenuation and exhibit a plaque burden of greater than or equal to 4%, then the patient would undergo the delipidation process of the present specification. In some alternate embodiments, it may be preferable to selectively modify HDL particles (and perform the HDL infusions) where the Fractional Flow Reserve (FFR) ranges from 80-100% and the accumulated lipid obstruction ranges from 20-70%, as noted by sections 404. It should be noted herein that in embodiments, a FFR measurement of 1-79% represents an ischemic condition, wherein a FFR measurement of 80-100% represents a non-ischemic condition. In most cases, treatment types ‘A’ and/or ‘B’ may be able to address the condition. Treatment type ‘D’ corresponds to cases where neither of the stated treatment types (A and/or B) is required. In some cells, such as cells 408, two treatment options may be indicated and the physician would decide upon the appropriate course of treatment.

Treatment type ‘C’ corresponds to cases where a combination of both a stent as well as selectively modified HDL particles are administered, as discussed below with reference to exemplary use case scenarios. Atherosclerosis is a systemic disease and patients may have multiple lesions throughout their vasculature. Therefore, it should be noted herein that the treatment methods of the present specification are not implemented based on an overall patient health-based treatment strategy, but rather a “lesion/plaque/area/region”-based treatment strategy. Thus, in a few cases, a physician may decide to combine the treatments and administer treatment type ‘C’ in addition to another treatment type. If, in a particular patient, one or more areas or lesions have a FFR of 79% or less (ranging from 1% to 79%), then those areas would have a stent implanted. If the same patient presents with additional, remaining lesions that are classified as low attenuation and exhibit a plaque burden of greater than or equal to 4%, then the patient would undergo a subsequent delipidation. Therefore, both interventions may be used for patients having multiple lesions with different levels of disease at each lesion. It should also be noted that after each delipidation treatment, a patient may be subjected to a diagnostic imaging (CCTA) to determine the efficacy of the treatment and whether additional treatment is warranted.

Lupus

Lupus is a systemic autoimmune disease whereby the body's immune system attacks its own tissues and organs. Lupus can cause inflammation, which can affect many of the body's systems, including joints, skin, kidneys, blood cells, brain, heart and lungs. Lupus may flare, become inactive (quiescent), or go into remission. In others, lupus may remain chronic, where people may experience frequent flares.

While lupus is difficult to diagnose, a combination of various blood and urine tests may be used to arrive at a differential diagnosis. These tests may include a complete blood count, an erythrocyte sedimentation rate, a kidney assessment, a liver assessment, a urinalysis, and/or an antinuclear antibody (ANA) test. Further, a chest X-ray or echocardiogram may be advised.

There is no standard treatment protocol for lupus as treatment depends on an individual's signs and symptoms, which vary greatly. Some common medications include:

Nonsteroidal anti-inflammatory drugs (NSAIDs). These include over-the-counter NSAIDs, such as naproxen sodium and ibuprofen, and prescription NSAIDs to treat pain, swelling and fever associated with lupus. Side effects of NSAIDs include stomach bleeding, kidney problems and an increased risk of heart problems.

Antimalarial drugs. Medications commonly used to treat malaria, such as hydroxychloroquine), affect the immune system and can help decrease the risk of lupus flares. Side effects can include an upset stomach.

Corticosteroids. Prednisone and other types of corticosteroids can counter the inflammation of lupus. High doses of steroids such as methylprednisolone are often used to control serious disease that involves the kidneys and brain. Side effects include weight gain, easy bruising, thinning bones (osteoporosis), high blood pressure, diabetes and increased risk of infection. The risk of side effects increases with higher doses and longer term therapy.

Immunosuppressants. Drugs that suppress the immune system may be helpful in serious cases of lupus and include azathioprine, mycophenolate mofetil, and methotrexate. Potential side effects may include an increased risk of infection, liver damage, decreased fertility and an increased risk of cancer.

Biologics. Intravenously administered belimumab may also reduce lupus symptoms in some people. Side effects include nausea, diarrhea and infections.

Rituximab can be beneficial in cases of resistant lupus. Side effects include infections and allergic reaction.

Lifestyle changes may also help prevent lupus flares and/or help one better cope with the symptoms including avoiding prolonged sun exposure; exercising regularly; avoiding smoking; eating healthy; and using vitamin and mineral supplements. Sometimes people with lupus seek alternative or complementary medicine. However, there aren't any alternative therapies that have been shown to alter the course of lupus, although some may help ease symptoms of the disease, and include DHEA supplementation; fish oil supplementation; and acupuncture. In addition, if left untreated or unmanaged, lupus can put one at risk of developing life-threatening problems such as a heart attack or stroke. In many cases, lupus nephritis does not cause any noticeable symptoms.

Because cardiovascular disease is so prevalent in, and the leading cause of death for many lupus patients, it is important to investigate and determine the presence of low attenuation plaque in patients with lupus. Lupus patients have a significantly higher burden (as many as ten times greater) of low attenuation plaque, in the coronary and/or carotid arteries, compared to their healthy counterparts, with the exception of those aged 60 years and older. Thus, lupus patients have a disproportionate share of low attenuation plaques that are at the highest risk of rupture due to their fatty and necrotic nature (i.e. cholesterol-filled). Low attenuation plaques may be present in both the lumen and embedded in the lumen wall of the arteries, which makes them particularly dangerous.

To date, most treatments, however, are aimed at suppressing or alleviating the symptoms of lupus, rather than addressing the associated lipid-related conditions, such as the buildup of low attenuation plaque. There is a need, therefore, for a comprehensive treatment protocol that may delay, halt and stabilize, reverse or improve the progression of diseases or pathophysiologic processes related to low attenuation plaque in patients with lupus.

In an embodiment, low-attenuation plaques may be defined as a plaque with a radiodensity below 30 Hounsfield units. In embodiments, a measurable minimum plaque size (as measured by CCTA) is defined as larger than 3 mm³ (for example, a cross-sectional area of about 3 mm² and a length of about 1 mm). In embodiments, candidates for the treatment protocols of the present specification have a greater than or equal to 4% obstruction that is entirely due to low attenuation plaque, where the percentage of obstruction refers to the amount of plaque present per vessel volume and is expressed as “plaque burden”. Therefore, in one embodiment, only patients who have a volume of low attenuation plaque per vessel volume greater than or equal to 4% shall receive the disclosed delipidation treatments.

In various embodiments, the methods and systems of the present specification effectively lowers the extent of low attenuation plaques by 10 to 50% and more specifically, by approximately 40%, as shown in post-treatment coronary CT angiogram scans (CTA) when compared with pre-treatment coronary CTA angiogram scans. In some embodiments, the methods and systems of the present specification effectively lower the extent of a patient's overall plaque burden to below 4%.

### Therapeutic Protocols for Administering Modified HDL Particles

In accordance with aspects of the present specification, treated plasma containing modified HDL particles with reduced lipid and/or increased pre-β concentration is administered to a patient in accordance with a plurality of therapeutic protocols. In some embodiments, therapy is based on a level of severity of the lipid-related condition, cardiac disease state, or low attenuation plaque volume, or plaque burden associated with lupus, as described above. In various embodiments, the plurality of therapy protocols comprises at least one or any combination of a plurality of therapeutic parameters such as, but not limited to:
Dosing range: 1 mg/kg to 250 mg/kg, and any increment therein, where a specific fixed dose may be calculated based on one or both of a patient's weight and the severity of the disease state.
Dosing volume: the average dosing volume is dependent upon the dose (in mg/kg) and the concentration of the product to be infused into the patient (treated plasma containing modified HDL particles or isolated pre-beta particles). In embodiments, the volume that is returned to the patient is substantially equal to the volume that was removed from the patient prior to the delipidation process. In embodiments, the volume that is returned to the patient is a concentrated volume. In embodiments, the volume delivered to a patient via infusion therapy is dependent upon the preparation of the product, whether it is treated plasma or concentrated, isolated pre-beta and the overall solubility of that product in a buffer or saline.
Dosing rate: the dose is provided via infusion therapy. It should be noted herein that the rate of infusion is the normal infusion rate for intravenous therapy, or 999 mL/hour and is thus dependent on overall volume and concentration. In an embodiment, the time of infusion ranges from one hour to eight hours.
Frequency or cycle of treatment: once per week for a duration of seven weeks, provided PDS-2 results on low attenuation plaques or plaque burden. Maintenance doses may be administered based on severity of disease and periodic cardiac imaging exams, such as coronary CT angiogram imaging exams (at least annually, or more frequently, such as semi-annually).
Duration or course of therapy: at least one day to at least one year

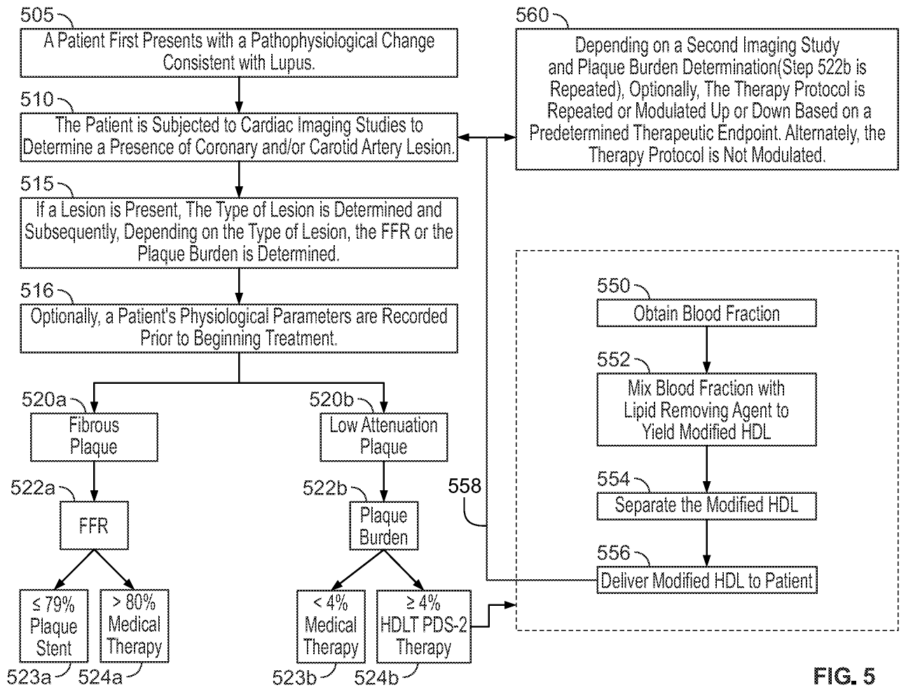

In embodiments, the treatment methods, protocols, and systems of the present specification are aimed at addressing the associated lipid-related conditions and/or cardiac conditions or disease states caused by lupus and/or lupus-driven inflammation, such as the buildup of low attenuation plaque and a minimum threshold plaque burden. FIG. 5 is a flowchart describing a plurality of exemplary steps of a therapy protocol for treating the associated lipid-related conditions of lupus, such as the buildup of low attenuation plaque or the presence of a minimum threshold plaque burden by stabilizing, reversing, or improving the progression of diseases or pathophysiologic processes related to the presence of low attenuation plaque in patients with lupus, in accordance with an embodiment of the present specification. At step 505, a patient first presents with a pathophysiological change that is consistent with lupus and diagnosed with lupus. Any of the aforementioned diagnostic techniques may be used in this step, including a complete blood count, an erythrocyte sedimentation rate, a kidney assessment, a liver assessment, a urinalysis, and/or an antinuclear antibody (ANA) test. At step 510, the patient is then subjected to any one or all of the following cardiac imaging studies to determine a presence of coronary and/or carotid artery obstruction or lesion: a chest X-ray, echocardiogram, ultrasound, CT scan, or CT angiogram (CCTA).

At step 515, the CT angiogram (CCTA) is analyzed and, if a lesion is present, the extent of obstruction or blockage (in terms of plaque burden or volume of low attenuation plaque), the nature or type of the lesion is determined and subsequently depending on the type of lesion the FFR or the plaque burden is determined. In a preferred embodiment, a CT angiogram is the standard imaging study that is used to determine both plaque burden and the extent of obstruction due to low attenuation plaque in the cardiovascular regions of a patient, including both the coronary and carotid arteries. CCTA has the ability to identify and differentiate carotid lesions, which are defined in terms of calcifications (large or speckled), presence of low-attenuation plaques, positive remodeling, and presence of napkin ring sign (NRS). This allows additional assessment of coronary plaque burden and plaque type, both of which may improve treatment modalities and outcomes of coronary atherosclerosis and cardiovascular disease states.

In an embodiment, the patient may present with a higher burden of low attenuation plaque as detected with a CT angiogram. Thus, a patient who is diagnosed with lupus is monitored to determine an extent of accumulation of low attenuation plaque in the coronary and/or carotid artery lumen, lumen walls, and any surrounding atherosclerotic plaque and the carotid artery lumen and any surrounding plaque, which can then be calculated into plaque burden. Because the low attenuation plaque can grow into the lumen wall, and not only the lumen itself, it is difficult to detect. Further to that, much of the initial plaque presents in the vessel wall rather than the vessel lumen itself, necessitating the ability to determine an extent of obstruction in the lumen and plaque burden on surrounding areas. In an embodiment, low-attenuation plaques may be defined as a plaque with a radiodensity below 30 Hounsfield units. In embodiments, the measurable plaque size is defined as larger than 3 mm³. In embodiments, the extent of plaque burden necessitating treatment is greater than or equal to 4%.

At optional step 516, one or more physiological parameters or biometrics of the patient are recorded. In embodiments, the one or more physiological parameters are those that may be incidental to determining one or more therapy parameters. For example, the patient's weight is recorded to determine a dosing range for the patient. It should be appreciated that the physiological parameters may be first recorded prior to step 505.

Based on the CCTA (of step 515), the identified lesion or plaque type is assessed as being fibrous plaque at step 520a or low attenuation plaque at step 520b. For fibrous plaque, FFR (Fractional Flow Reserve) is used as a parameter at step 522a to determine a type of treatment/intervention. If the FFR is less than or equal to 79% an invasive treatment is recommended, at step 523a, where a stent is embedded through physical intervention. If the FFR is greater than 80% an appropriate medical therapy is recommended at step 524a. That is, the patient may be treated with medications such as, for example, statins.

On the other hand, for low attenuation plaque, plaque burden is used as a parameter at step 522b to determine a type of treatment/intervention. If the plaque burden is less than a predefined target/threshold of 4% an appropriate medical therapy is recommended at step 523b. However, if the plaque burden is greater than or equal to the predefined target/threshold of 4% the patient is administered modified HDL particle-based therapy (that is, a delipidation process) of the present specification, at step 524b.

The modified HDL particle-based therapy or delipidation process includes the following steps:

First, a blood fraction of the patient is obtained at step 550. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 5. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, blood fraction obtained at 120 includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

Secondly, at step 552, the blood fraction obtained is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles. Energy is introduced into the system in the form of varied mixing methods, time, and speed.

Thirdly, at step 554, bulk solvents are removed from the modified HDL particles via centrifugation. In embodiments, any remaining soluble solvent is removed via charcoal adsorption, evaporation, or Hollow Fiber Contractors (HFC) pervaporation. The mixture is optionally tested for residual solvent via use of chromatography (GC), or similar means. The test for residual solvent may optionally be eliminated based on statistical validation.

Finally, at step 556, the treated plasma containing modified HDL particles with reduced lipid content, which was separated from the solvents, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In an embodiment, the therapy protocol comprises an infusion delivery of modified HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour+/−100 ml/hour or a rate deemed more appropriate for the patient. In embodiments, the treatment is repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of modified HDL particles over a predetermined time period at a rate of 999 mL/hour.

In embodiments, the patient is monitored again, at step 558, for changes in the previously monitored atheroma areas and volumes, specifically for low attenuation plaque having a plaque burden of greater than or equal to 4%. The process repeats from step 522b and if it is determined that there is still a plaque burden greater than or equal to 4%, at optional step 560, the therapy protocol is repeated or may be titrated or modulated up or down based on a therapeutic endpoint. In embodiments, the treatment protocols, methods, and systems of the present specification effectively lowers low attenuation plaques by 10 to 50% and more specifically, by approximately 40%, as shown in post-treatment CT angiogram scans when compared with pre-treatment CT angiogram (CTA) scans. In some embodiments, the methods and systems of the present specification effectively lower the extent of a patient's overall plaque burden to below 4%. In embodiments, one or more intra-treatment assessments are made to determine a level of improvement or therapeutic effect. In an embodiment, an assessment may include a CT angiogram scan. The one or more intra-treatment assessments are made at predetermined points in time during the course of therapy.

If the intra-treatment severity level assessments show a delay in the onset of additional symptoms, a halting in the worsening of symptoms, or an improvement in the patient's condition, it is considered to be of therapeutic benefit.

In embodiments, when therapeutic benefit is shown, the therapeutic amount may be titrated down wherein parameters such as, but not limited to, the dose range, frequency or cycle of treatment and/or course of therapy may be reduced. Alternately, the therapy protocol may be titrated up depending on various factors.

Still alternately, if the intra-treatment severity level assessments show or do not show improvement in the patient's condition, the therapy protocol is not modulated.

By way of example, for a lupus patient weighing 100 kg, where a dosage of ApoA-I is determined to be 15 mg/kg, that patient will receive a dose of 1.5 g. It should be noted that if the patient presents with mild, moderate or severe obstruction of a coronary and/or carotid artery, that dosage may be increased. The overall volume delivered to the patient via infusion therapy depends on the therapeutic product that is solubilized in a buffer or saline. For example, if the therapeutic product is autologous treated plasma, then the patient will receive a volume of therapeutic product equivalent to the volume that was extracted from the patient. If the therapeutic product is non-autologous treated plasma, the patient may receive a volume of 1 L as one example. If the therapeutic product is non-autologous isolated, concentrated pre-beta particles, the volume may be much lower.

### Therapeutic Endpoints/Objectives for Treating Lupus

In various embodiments, a lupus patient's baseline level of cardiac disease is diagnosed/assessed and categorized as, one of early onset, mild, moderate or severe as described above. The baseline, starting or initial severity level refers to the severity of the associated lipid-related condition or cardiac disease caused by lupus, such as the buildup of low attenuation plaque in at least one of the coronary and/or carotid arteries (lumen and lumen walls), as measured by cardiac imaging and as expressed by plaque burden, before the patient is treated with the modified HDL and/or isolated pre-β HDL therapy of the present specification.

In embodiments, the baseline, starting or initial severity level is diagnosed/assessed using at least one physiological diagnostic or advanced medical imaging technique. In embodiments, coronary CT angiogram is used. It should be noted that efficacy of treatment is measured by comparing a patient's CT angiogram pre-treatment to a CT angiogram post-treatment. In embodiments, the physiological parameter refers to information that can be ascertained from a coronary CT angiogram. In embodiments, the physiological parameter is the percentage of blockage or obstruction of a patient's coronary and/or carotid artery. In embodiments, the physiological parameter is the amount or volume of low attenuation plaque in a patient's coronary and/or carotid artery in both the lumen and lumen walls. In embodiments, the physiological parameter is the amount of low attenuation plaque in a patient's coronary and/or carotid artery in both the lumen and lumen walls. In embodiments, the physiological parameters is measured as a plaque burden.

In an embodiment, a therapeutic benefit is recognized when a patient is able to maintain or be stabilized in their current state when treated with a therapy protocol of the present specification.

In an embodiment, a therapeutic benefit is recognized when a patient maintains/stabilizes with respect to a disease or pathophysiologic process in cardiovascular conditions associated with lupus and/or a lupus-associated lipid-related condition when treated with a therapy protocol of the present specification when compared to a placebo or an untreated person.

In an embodiment, a therapeutic benefit is recognized when a patient shows a delay or halting of worsening of a disease or pathophysiologic process in cardiovascular conditions associated with lupus and/or a lupus-associated lipid-related condition when treated with a therapy protocol of the present specification when compared to a placebo or an untreated person.

In an embodiment, a therapeutic benefit is recognized when a patient shows a delay in the rate of progression of a disease or pathophysiologic process in cardiovascular conditions associated with lupus and/or a lupus-associated lipid-related condition when treated with a therapy protocol of the present specification when compared to a placebo or an untreated person.

In an embodiment, a therapeutic benefit is recognized when a patient shows an improvement in a disease or pathophysiologic process in cardiovascular conditions associated with lupus and/or a lupus-associated lipid-related condition when treated with a therapy protocol of the present specification when compared to a placebo or an untreated person.

In some embodiments, after at least one treatment session or determinable time period at the end of which an effect of said at least one treatment session is amenable to measurement, the patient experiences a decrease in the accumulation of low attenuation plaque in the lumen of a coronary or carotid artery or a lumen wall of a coronary or carotid artery.

In some embodiments, after at least one treatment session or determinable time period at the end of which an effect of said at least one treatment session is amenable to measurement, the patient experiences a decrease of a plaque burden in the lumen of a coronary or carotid artery or a lumen wall of a coronary or carotid artery.

In some embodiments, after at least one treatment session or determinable time period at the end of which an effect of said at least one treatment session is amenable to measurement, the patient experiences a decrease in plaque burden to less than 4% in the lumen of a coronary or carotid artery or a lumen wall of a coronary or carotid artery.

Following are a plurality of non-limiting, exemplary therapeutic endpoints with reference to the baseline, starting or initial severity level:

In embodiments, after at least one treatment session or determinable time period at the end of which an effect of said at least one therapy treatment is amenable to measurement, the rate of progression, level or amount of a patient's physiological parameter, is unchanged relative to the rate, level or amount of that patient's physiological parameter before therapy treatment.

In embodiments, after at least one treatment session or determinable time period at the end of which an effect of said at least one therapy treatment is amenable to measurement, the rate of progression, level or amount of a patient's physiological parameter, is delayed relative to the rate, level or amount of that patient's physiological parameter before therapy treatment.

In embodiments, after at least one treatment session or determinable time period at the end of which an effect of said at least one therapy treatment is amenable to measurement, the rate of progression, level or amount of a patient's physiological parameter, is modified relative to the rate of progression, level or amount of that patient's physiological parameter before therapy treatment.

In embodiments, after at least one treatment session or determinable time period at the end of which an effect of said at least one therapy treatment is amenable to measurement, the rate of progression, level or amount of that patient's physiological parameter is improved relative to the rate of progression, level or amount of that patient's physiological parameter before therapy treatment.

In some embodiments, after at least one treatment session or determinable time period at the end of which an effect of said at least one treatment session is amenable to measurement, the patient experiences a decrease in the accumulation of low attenuation plaque in a coronary and/or carotid artery (lumen or lumen wall) space indicative of an improvement or stabilization of lupus-related symptoms.

In some embodiments, after at least one treatment session or determinable time period at the end of which an effect of said at least one treatment session is amenable to measurement, the patient experiences a decrease in plaque burden in a coronary and/or carotid artery (lumen or lumen wall) space indicative of an improvement or stabilization of lupus-related symptoms.

In some embodiments, after at least one treatment session or determinable time period at the end of which an effect of said at least one treatment session is amenable to measurement, the patient experiences a decrease to less than 4% of plaque burden in a coronary and/or carotid artery (lumen or lumen wall) space indicative of an improvement or stabilization of lupus-related symptoms.

Cardiovascular Diseases

Familial Hypercholesterolemia (FH) is an inherited genetic autosomal dominant disease characterized by markedly elevated low-density lipoprotein (LDL), tendon xanthomas, and premature coronary heart disease, caused by mutations of “FH genes,” which include the LDL-receptor (LDLR), apolipoprotein B-100 (APOB) or proprotein convertase subtilisin/kexin type 9 (PCSK9). FH produces a clinically recognizable pattern that consists of severe hypercholesterolemia due to the accumulation of LDL in the plasma, cholesterol deposition in tendons and skin, as well as a high risk of atherosclerosis manifesting almost exclusively as coronary artery disease (CAD). In FH patients, this genetic mutation makes the liver unable to effectively metabolize (or remove) excess plasma LDL, resulting in increased LDL levels.

If an individual has inherited a defective FH gene from one parent, the form of FH is called Heterozygous FH. Heterozygous FH is a common genetic disorder, inherited in an autosomal dominant pattern, occurring in approximately 1:500 people in most countries. If the individual has inherited a defective FH gene from both parents, the form of FH is called Homozygous FH. Homozygous FH is very rare, occurring in about 1 in 160,000 to one million people worldwide, and results in LDL levels >700 mg/dl, 10-fold higher than the ideal 70 mg/dl level desired for patients with CVD. Due to the high LDL levels, patients with Homozygous FH have aggressive atherosclerosis (narrowing and blocking of blood vessels) and early heart attacks. This process starts before birth and progresses rapidly. It can affect the coronary arteries, carotid arteries, aorta, and aortic valve.

Heterozygous FH (HeFH) is normally treated with statins, bile acid sequestrants, or other lipid lowering agents that lower cholesterol levels, and/or by offering genetic counseling. Homozygous FH (HoFH) often does not respond adequately to medical therapy and may require other treatments, including LDL apheresis (removal of LDL in a method similar to dialysis), ileal bypass surgery to dramatically lower their LDL levels, and occasionally liver transplantation. A few medications have recently been approved for use by HoFH subjects. However, these medications lower LDL only, and modestly contribute to slowing, but not stopping, further progression of atherosclerosis. Additionally, these medications are known to have significant side-effects.

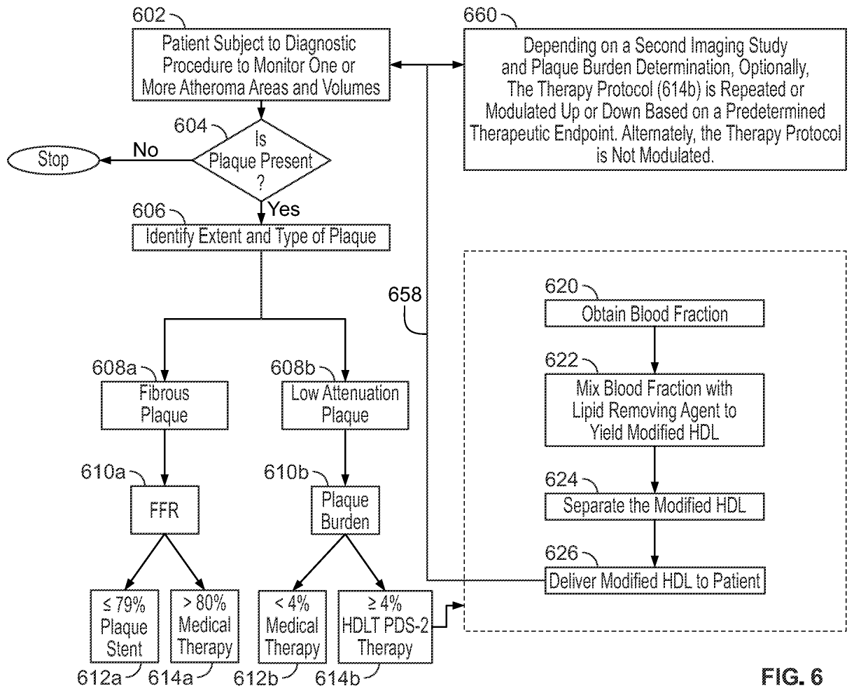

FIG. 6 is a flow chart illustrating an exemplary process of diagnosing and treating cardiovascular diseases, such as, but not limited to homozygous familial hypercholesterolemia (HoFH), heterozygous familial hypercholesterolemia (HeFH), Ischemic stroke, Coronary Artery Disease (CAD), Acute Coronary Syndrome (ACS), and peripheral arterial disease (PAD), in accordance with some embodiments of the present specification. At step 602, a subject or a patient who is diagnosed with a cardiovascular disease is monitored for one or more atheroma areas and/or volumes via a diagnostic procedure. In an embodiment, advanced medical imaging techniques, such as, but not limited to Computed Tomography (CT) angiogram and/or Intravascular Ultrasound (IVUS), may be used to detect areas within the inner layer of artery walls where plaque may have accumulated. Plaque may swell in the artery wall, thereby intruding into the channel of the artery and narrowing it, resulting in restriction of blood flow. In some embodiments, the plaque may include low attenuation plaque, which comprises necrotic, fatty, or degenerative material, including macrophage cells or debris, lipids, and/or calcium. In some embodiments, the plaque may include a variable amount of fibrous connective tissue (fibrous or fibromuscular plaque). Analysis from the imaging techniques may be used to identify and therefore monitor both the type of and volumes of plaque accumulated within the inner layer of artery walls.

At step 604 it is determined, based on analysis from the diagnostic technique of step 602 if plaque or plaque burden is detected. If no plaque or plaque burden is detected, or if the level of plaque or plaque burden falls outside a pre-determined range of values, the process is stopped. However, if plaque or plaque burden is detected then the flow moves to step 606 wherein the extent and type of plaque is determined.

Based on diagnostic step 602, the identified lesion or plaque type is assessed as being fibrous plaque at step 608a or low attenuation plaque at step 608b. For fibrous plaque, FFR (Fractional Flow Reserve) is used as a parameter at step 610a to determine a type of treatment/intervention. In an embodiment, FFR is used to measure pressure differences across a coronary artery stenosis to determine the likelihood that the stenosis impedes blood oxygen delivery to the heart muscle (ischemia). In an embodiment, the physician identifies one or more arteries with stenosis that have a blockage of 20%-70% due to accumulated lipids, in order to implement at least one treatment method in accordance with the present specification. If the FFR is less than or equal to 79% an invasive treatment is recommended, at step 612a, where a stent is embedded through physical intervention. If the FFR is greater than 80% an appropriate medical therapy is recommended at step 614a. That is, the patient may be treated with medications such as, for example, statins.

In a first optional scenario, a physician determines whether the amount of accumulated lipid-containing material, covering a lesion/plaque/area/region, falls above a predetermined threshold value or within a range of values, as measured in terms of a percentage of blockage due to lipid content. If arteries with atheroma lesion(s) having an amount or volume of lipid-containing material above the threshold percentage value or that fall within a range of values are not identified, an alternative treatment process (which may include no treatment or physical intervention) is determined by the physician. If arteries with lipid-containing atheroma lesion/plaque/area/region(s) having an amount or volume of lipid blockage above the predetermined threshold percentage or within a predetermined range of percentages are identified, the patient is then subjected to the delipidation process. The delipidation process of the present specification is described in greater detail below.

In a second optional scenario, a physician determines whether, based on the FFR measurement, blood oxygen delivery is impeded below a threshold value or within a range of values (which is expressed as the maximal flow of blood down a vessel in the presence of stenosis compared to the maximal flow in the hypothetical absence of stenosis). If blood oxygen delivery is impeded below a threshold value or within a predetermined range of values, then a physician treats with physical intervention, such as a stent. However, if it is determined that blood oxygen delivery is not impeded below a threshold value or does not fall within a predetermined range of values, the physician explores an alternate treatment option (which may include no treatment or the delipidation process of the present specification). In an embodiment, the threshold value is 80%. In an embodiment, the range of values is 1%-79%.

In a third optional scenario, a physician determines whether both the accumulated lipid-containing material, covering a lesion/plaque/area/region(s) is of an amount or volume falling within a predetermined range of percentages and blood oxygen delivery is impeded as determined by a predetermine range of percentages. If both conditions are met the physician treats those areas identified as ischemic areas (FFR measurement in a range of 1% to 79%, and preferably below 80%) with a stent implant procedure and subsequently, the remaining areas with the delipidation process of the present specification. However, if both threshold conditions are not met, then the physician determines if either one of the conditions or neither condition is met and determines an appropriate course of treatment as outlined above.

In an example case, where the analysis from the imaging determines a FFR in the range of 1%-79%, and blockage due to lipids anywhere from 1 to 100%, a physician may decide to physically intervene to improve the blood flow as measured by FFR, and thus, blood oxygen delivery. In an embodiment, the physical intervention is performed by surgically embedding a stent in order to increase the rate of blood flow in the identified atheroma area.

In another example, where the analysis from the imaging determines a FFR in the range of 80%-100%, and blockage due to lipids to be in the range of 20%-70%, the physician may opt for treatment methods that remove or reduce the lipids. In this example, embodiments of the present specification that enable selective modification of HDL particles are utilized.

In yet another example, where the FFR is determined to be in a range of 1% to 79%, and preferably less than 80%, and blockage due to lipids is in the range of 20%-70%, the physician may opt to proceed with the surgical process of embedding a stent. It should be appreciated that when a percentage blockage is stated, such as 20%-70%, it means that a cross-sectional area of a vessel is blocked with lipid containing material and that such blockage occupies a range of 20% to 70% of the cross-sectional area of the vessel.

For low attenuation plaque, assessed at the previous step 608b, plaque burden is used as a parameter at step 610b to determine a type of treatment/intervention. If the plaque burden is less than a predefined target/threshold of 4% an appropriate medical therapy is recommended at step 612b. However, if the plaque burden is greater than or equal to the predefined target/threshold of 4% the patient is administered modified HDL particle-based therapy (that is, a delipidation process) of the present specification, at step 614b. Stated differently, if arteries with lipid-containing atheroma lesion/plaque/area/region(s) having an amount or volume of plaque burden greater than or equal to 4% are identified, the patient is then subjected to the delipidation process. The delipidation process includes the following steps:

At step 620, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 6. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, blood fraction obtained includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

At step 622, the blood fraction obtained at step 620 is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles.

Energy is introduced into the system in the form of varied mixing methods, time, and speed. At step 624, bulk solvents are removed from the modified HDL particles via centrifugation. In embodiments, any remaining soluble solvent is removed via charcoal adsorption, evaporation, or Hollow Fiber Contractors (HFC) pervaporation. The mixture is optionally tested for residual solvent via use of chromatography (GC), or similar means. The test for residual solvent may optionally be eliminated based on statistical validation.

At step 626, the treated plasma containing modified HDL particles with reduced lipid content, which was separated from the solvents at step 624, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In embodiments, the patient is monitored again, in step 658, for changes in the previously monitored atheroma areas and volumes, specifically for low attenuation plaque having a plaque burden of greater than or equal to 4%. If low attenuation plaque having a plaque burden of greater than or equal to 4% is present, at step 660, the process is repeated from step 614b, as described above. In embodiments, the patient is monitored repeatedly within a period of three to six months. The treatment cycle is also repeated at this frequency until the monitoring suggests substantially or completely enhanced cholesterol efflux. In an embodiment, when the atheroma area and volume are monitored to be below threshold, the patient may be considered to have been treated and may not require further repetition of the treatment cycle. In some embodiments, frequency of treatment may vary depending on the volume to be treated and the severity of the condition of the patient.

In some embodiments, after the delipidation process—that is, after administering to the patient the high density lipoprotein composition, the patient experiences a reduction of the accumulation of plaque in an artery supplying blood to the patient's heart.

In some embodiments, after the delipidation process—that is, after administering to the patient the high density lipoprotein composition, the patient experiences a decrease in the plaque burden in an artery supplying blood to the patient's heart.

In some embodiments, after the delipidation process—that is, after administering to the patient the high density lipoprotein composition, the patient experiences a decrease in the plaque burden, to lower that 4%, in an artery supplying blood to the patient's heart.

In some embodiments, after the delipidation process—that is, after administering to the patient the high-density lipoprotein composition, a percentage of obstruction of the patient's physiological parameters, indicative of the lipid-related condition, stabilizes and does not experience a further increase in obstruction.

Atheroembolic Renal Disease (AERD)/Renal Arterial Stenosis (RAS)/Kidney Transplant/and CardioRenal Syndrome (CRS)

The cardiac and renal systems are closely related in that there are hemodynamic interactions of the heart and kidney in heart failure, cytokines, atherosclerotic disease conditions across both organ systems, and structural changes in the heart unique to kidney disease progression. Cardiorenal syndrome (CRS) encompasses a spectrum of disorders involving both the heart and kidneys in which acute or chronic dysfunction in one organ may induce acute or chronic dysfunction in the other organ.

Renal Arterial Stenosis (RAS) refers to a blockage in an artery that supplies blood to the kidney and is characterized in two forms: a) smooth muscle plaque (fibrous or fibro-muscular plaque) and/or b) low-attenuation or lipid-filled plaque. This condition, generally known as renal arterial stenosis, decreases blood flow to the kidney and can result in high blood pressure. Both fibrous and low-attenuation plaque in the renal arteries may be discovered during a CT angiogram. In some cases, renal arterial stenosis is discovered while performing a CT angiogram for an aortic aneurysm. While blood pressure typically increases gradually with age, a sudden onset of hypertension is also likely to be associated with renal obstruction or renal arterial stenosis. A decrease in flow of blood to the kidney causes vasoconstriction or high blood pressure, as the kidney starts producing an excess of cytokines.

In addition, a “cholesterol embolism’, can occur when the cholesterol in the artery is released, usually from a low attenuation atherosclerotic plaque and travels as an embolus in the bloodstream causing an obstruction (as an embolism) in blood vessels that are positioned further away. Once in circulation, the cholesterol particles get stuck in tiny blood vessels, or arterioles. They can reduce blood flow to tissues and cause inflammation and tissue damage that can harm the kidneys. A cholesterol embolism may result in renal failure and is a disease state referred to as Atheroembolic Renal Disease (AERD). AERD is one of the manifestations of diseases that may occur due to a lipid-filled or low attenuation plaque. In a patient with AERD, the plaque may rupture in the artery and release the cholesterol and other “junk” within the plaque into the vessel. The released cholesterol and junk may travel down the artery and may block the artery and injure a part of the kidney and its tissues, thereby resulting in AERD. Atherosclerosis of the aorta is the most common cause of AERD.

Currently, treatment of RAS, its manifestations such as AERD, and other cardiovascular diseases involves putting a stent in an artery to open the vessel. However, installing a stent is likely to only treat the symptoms, such as by normalizing high blood pressure. There are also instances when the blood pressure is normal, but AERD is present in a patient. There is thus a need to address the underlying cause of the disease and treat RAS either in combination with or independently of high blood pressure symptoms.

Because RAS is a systemic disease, patients may have multiple lesions throughout their vasculature. Sometimes, the plaque within the arteries may break away and damage kidneys, resulting in Atheroembolic Renal Disease (AERD). Patients with kidney damage show marked decrease in renal function, which is further classified into acute kidney disease and chronic kidney disease based on the difference in the degree to which the renal function is impaired. Specifically, chronic kidney disease refers to kidney disease in which decreased renal function chronically persists for 3 months or more. Chronic kidney disease is considered to constitute a public health threat due to the high risk of progressing to end-stage kidney disease. There is no effective treatment for chronic kidney disease, and when chronic kidney disease progresses resulting in decreased renal function, there is the risk of uremia resulting in the need for dialysis or kidney transplant, which are considerably burdensome in terms of medical care economics. There are approximately 125,000 kidney transplant patients in the U.S. alone. The treatment methods of the present specification are directed towards providing effective therapy for such patients of end-stage kidney disease and in need of kidney transplant. It should be noted herein that the treatment methods of the present specification are not implemented based on an overall patient health-based treatment strategy, but rather a “lesion/plaque/area/region”-based treatment strategy.

Different types of treatments may be provided depending on the diagnostic results and threshold values. At this stage, the physician may determine that either the treatment in accordance with embodiments of the present specification is not required as the disease has subsided, is not present, is not sufficient, or has been treated; or an alternative form of treatment is required.

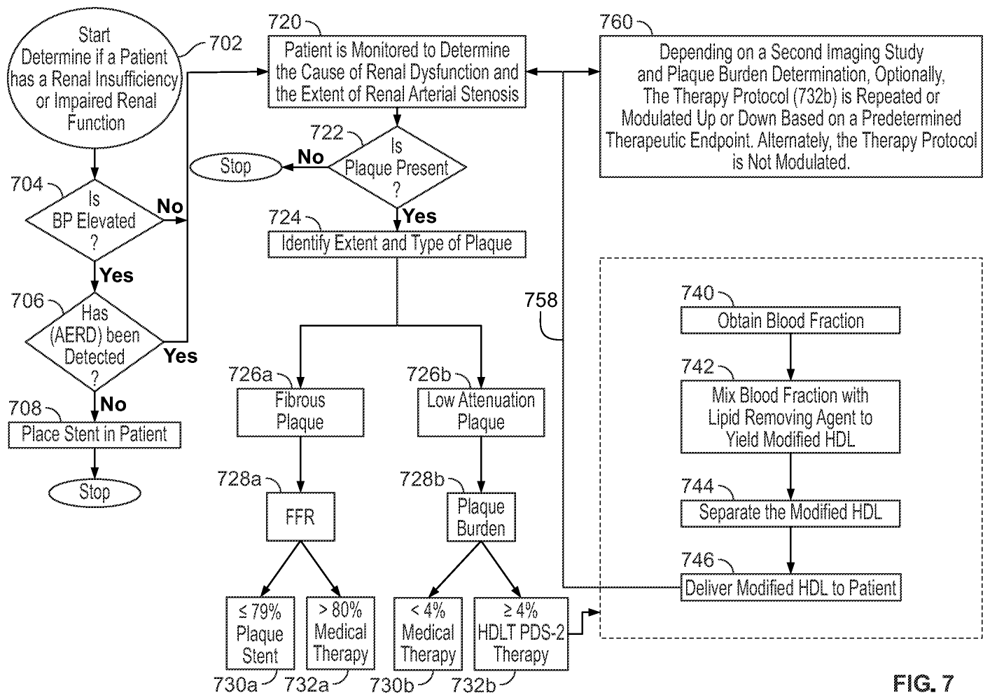

FIG. 7 is a flow chart illustrating an exemplary process of treating lipid-related diseases, such as, but not limited to CRS, RAS, AERD and end-stage kidney disease (leading to kidney transplant), in accordance with some embodiments of the present specification. By way of example, at step 702, a patient first presents with renal insufficiency or impaired renal function.—In some embodiments, the impaired renal function may be due to a blockage, such as plaque, in an artery that supplies blood to the kidney. The plaque may swell in the artery wall, thereby intruding into the channel of the artery and narrowing it, resulting in restricting of blood flow and causing renal abnormalities.

At step 704, it is determined whether a patient has elevated Blood Pressure (BP). Recent onset of hypertension may be a clinical manifestation of the presence of plaque. If it is determined that the patient has High BP (HBP), the physician, may look for atheroembolic renal disease (AERD) at step 706. While AERD may not cause any symptoms, some of the following symptoms may appear slowly and worsen over time: blood in the urine, fever, muscle aches, headache, weight loss, foot pain or blue toes, nausea, among other symptoms. If AERD is not identified, then at 708, a stent is placed in the patient to reverse any blockage that may be resulting in HBP. If at step 704, it is determined that the patient does not have elevated BP then the flow moves to step 720. Also, if at step 706, AERD is not detected then the process also moves to step 720.

At step 720, the patient is monitored to determine the cause of renal dysfunction and the extent of renal arterial stenosis via a diagnostic procedure. In an embodiment, advanced medical imaging techniques, such as, but not limited to Computed Tomography (CT) angiogram and/or Intravascular Ultrasound (IVUS), may be used to detect areas within the inner layer of artery walls where plaque may have accumulated. Plaque may swell in the artery wall, thereby intruding into the channel of the artery and narrowing it, resulting in restriction of blood flow. In some embodiments, the plaque may include low attenuation plaque, which comprises necrotic, fatty, or degenerative material, including macrophage cells or debris, lipids, and/or calcium. In some embodiments, the plaque may include a variable amount of fibrous connective tissue (fibrous or fibromuscular plaque). Analysis from the imaging techniques may be used to identify and therefore monitor both the type of and volumes of plaque accumulated within the inner layer of artery walls.

At step 722 it is determined, based on analysis from the diagnostic technique of step 720 if plaque or plaque burden is detected. If no plaque or plaque burden is detected, or if the level of plaque or plaque burden falls outside a pre-determined range of values, the process is stopped. However, if plaque or plaque burden is detected then the flow moves to step 724 wherein the extent and type of plaque is determined.

Based on the diagnostic step 720, the identified lesion or plaque type is assessed as being fibrous plaque at step 726a or low attenuation plaque at step 726b. For fibrous plaque, FFR (Fractional Flow Reserve) is used as a parameter at step 728a to determine a type of treatment/intervention. In an embodiment, FFR is used to measure pressure differences across a renal artery stenosis to determine the likelihood that the stenosis impedes blood oxygen delivery to the kidney. In an embodiment, the physician identifies one or more arteries with stenosis that have a blockage of 20%-70% due to accumulated lipids, in order to implement at least one treatment method in accordance with the present specification. If the FFR is less than or equal to 79% an invasive treatment is recommended, at step 730a, where a stent is embedded through physical intervention. If the FFR is greater than 80% an appropriate medical therapy is recommended at step 732a. That is, the patient may be treated with medications such as, for example, statins.

In a first optional scenario, a physician determines whether the amount of accumulated lipid-containing material, covering a lesion/plaque/area/region, falls above a predetermined threshold value or within a range of values, as measured in terms of a percentage of blockage due to lipid content. If arteries with lesion(s) having an amount or volume of lipid-containing material above the threshold percentage value or that fall within a range of values are not identified, an alternative treatment process (which may include no treatment or physical intervention) is determined by the physician. If arteries with lipid-containing lesion/plaque/area/region(s) having an amount or volume of lipid blockage above the predetermined threshold percentage or within a predetermined range of percentages are identified, the patient is then subjected to the delipidation process. The delipidation process of the present specification is described in greater detail below.

In a second optional scenario, a physician determines whether, based on the FFR measurement, blood oxygen delivery is impeded below a threshold value or within a range of values (which is expressed as the maximal flow of blood down a vessel in the presence of stenosis compared to the maximal flow in the hypothetical absence of stenosis). If blood oxygen delivery is impeded below a threshold value or within a predetermined range of values, then a physician treats with physical intervention, such as a stent. However, if it is determined that blood oxygen delivery is not impeded below a threshold value or does not fall within a predetermined range of values, the physician explores an alternate treatment option (which may include no treatment or the delipidation process of the present specification). In an embodiment, the threshold value is 80%. In an embodiment, the range of values is 1%-79%.

In a third optional scenario, a physician determines whether both the accumulated lipid-containing material, covering a lesion/plaque/area/region(s) is of an amount or volume falling within a predetermined range of percentages and blood oxygen delivery is impeded as determined by a predetermine range of percentages. If both conditions are met the physician treats those areas identified as ischemic areas (FFR measurement in a range of 1% to 79%, and preferably below 80%) with a stent implant procedure and subsequently, the remaining areas with the delipidation process of the present specification. However, if both threshold conditions are not met, then the physician determines if either one of the conditions or neither condition is met and determines an appropriate course of treatment as outlined above.

In an example case, where the analysis from the imaging determines a FFR in the range of 1%-79%, and blockage due to plaque anywhere from 1 to 100%, a physician may decide to physically intervene to improve the blood flow as measured by FFR, and thus, blood oxygen delivery. In an embodiment, the physical intervention is performed by surgically embedding a stent in order to increase the rate of blood flow in the identified plaque area.

In another example, where the analysis from the imaging determines a FFR in the range of 80%-100%, and blockage due to lipids to be in the range of 20%-70%, the physician may opt for treatment methods that remove or reduce the lipids. In this example, embodiments of the present specification that enable selective modification of HDL particles are utilized.

In yet another example, where the FFR is determined to be in a range of 1% to 79%, and preferably less than 80%, and blockage due to lipids is in the range of 20%-70%, the physician may opt to proceed with the surgical process of embedding a stent. It should be appreciated that when a percentage blockage is stated, such as 20%-70%, it means that a cross-sectional area of a vessel is blocked with lipid containing material and that such blockage occupies a range of 20% to 70% of the cross-sectional area of the vessel.

For low attenuation plaque, assessed at the previous step 726b, plaque burden is used as a parameter at step 728b to determine a type of treatment/intervention. If the plaque burden is less than a predefined target/threshold of 4% an appropriate medical therapy is recommended at step 730b. However, if the plaque burden is greater than or equal to the predefined target/threshold of 4% the patient is administered modified HDL particle-based therapy (that is, a delipidation process) of the present specification, at step 732b. Stated differently, if arteries with lipid-containing lesion/plaque/area/region(s) having an amount or volume of plaque burden greater than or equal to 4% are identified, the patient is then subjected to the delipidation process. The delipidation process includes the following steps:

At step 740, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 7. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, blood fraction obtained includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

At step 742, the blood fraction obtained at step 740 is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles.

Energy is introduced into the system in the form of varied mixing methods, time, and speed. At step 744, bulk solvents are removed from the modified HDL particles via centrifugation. In embodiments, any remaining soluble solvent is removed via charcoal adsorption, evaporation, or Hollow Fiber Contractors (HFC) pervaporation. The mixture is optionally tested for residual solvent via use of chromatography (GC), or similar means. The test for residual solvent may optionally be eliminated based on statistical validation.

At step 746, the treated plasma containing modified HDL particles with reduced lipid content, which was separated from the solvents at step 744, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In embodiments, the patient is monitored again, in step 758, for changes in the previously monitored atheroma areas and volumes, specifically for low attenuation plaque having a plaque burden of greater than or equal to 4%. If low attenuation plaque having a plaque burden of greater than or equal to 4% is present, at step 760, the process is repeated from step 732b, as described above.

In embodiments, the patient is monitored again for changes in the previously monitored atheroma areas and volumes, specifically for low attenuation plaque having a plaque burden of greater than or equal to 4%. Therefore, the process is repeated from step 720, as described above.

In embodiments, the patient is monitored repeatedly within a period of three to six months. The treatment cycle is also repeated at this frequency until the monitoring suggests substantially or completely enhanced cholesterol efflux. In an embodiment, when the plaque area and volume are monitored to be below threshold, the patient may be considered to have been treated and may not require further repetition of the treatment cycle. In some embodiments, frequency of treatment may vary depending on the volume to be treated and the severity of the condition of the patient.

In some embodiments, after the delipidation process—that is, after administering to the patient the high density lipoprotein composition, the patient experiences a reduction of the accumulation of plaque in an artery supplying blood to the patient's kidney.

In some embodiments, after the delipidation process—that is, after administering to the patient the high density lipoprotein composition, the patient experiences a decrease in the plaque burden in an artery supplying blood to the patient's kidney.

In some embodiments, after the delipidation process—that is, after administering to the patient the high density lipoprotein composition, the patient experiences a decrease in the plaque burden, to lower that 4%, in an artery supplying blood to the patient's kidney.

In some embodiments, after the delipidation process—that is, after administering to the patient the high density lipoprotein composition, a percentage of obstruction of the patient's physiological parameters, indicative of the lipid-related condition, stabilizes and does not experience a further increase in obstruction.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for delaying a progression of, stabilizing, or improving at least one lipid-related condition in a patient, comprising:
determining a type of plaque present in at least one artery of the patient, wherein said plaque may be found within the lumen of the at least one artery, wherein said type of plaque is at least one of a fibrous plaque or a low attenuation plaque;
if, based on said determining, the plaque is a low attenuation plaque, subsequently determining a volume of the low attenuation plaque;
calculating a plaque burden using the volume of low attenuation plaque;
administering a delipidation treatment protocol for the patient if the plaque burden is greater than or equal to a predetermined threshold value, wherein the delipidation treatment protocol comprises acquiring a blood fraction from the patient, mixing said blood fraction with a lipid removing agent to yield a delipidated blood fraction, deriving a high-density lipoprotein composition from the delipidated blood fraction, and administering to the patient the high-density lipoprotein composition.

2. The method of claim 1, wherein the at least one lipid-related condition is lupus and wherein the low attenuation plaque is determined based on monitoring of a pathophysiological change indicative of lupus.

3. The method of claim 2, wherein, after administering to the patient the high-density lipoprotein composition, a percentage of obstruction of the patient's physiological parameters indicative of lupus stabilizes and does not experience a further increase in obstruction.

4. The method of claim 1, wherein the high-density lipoprotein composition derived from mixing the blood fraction with the lipid removing agent is delivered to the patient via infusion therapy in a dosage ranging from 1 mg/kg to 250 mg/kg.

5. The method of claim 1, wherein the high-density lipoprotein composition derived from mixing the blood fraction of the patient with the lipid removing agent is delivered to the patient via infusion therapy at a rate of 999 mL per hour plus or minus 100 mL per hour.

6. The method of claim 1, wherein, after administering to the patient the high-density lipoprotein composition, the patient experiences a regression of the accumulation of low attenuation plaque in the at least one artery.

7. The method of claim 1, wherein, after administering to the patient the high-density lipoprotein composition, the patient experiences a reduction of plasma burden in the at least one artery.

8. The method of claim 1, further comprising:
connecting the patient to a device for withdrawing blood;
withdrawing blood from the patient; and
separating blood cells from the blood to yield the blood fraction containing high density lipoproteins and low density lipoproteins.

9. The method of claim 1, wherein the at least one lipid-related condition is atheroembolic renal disease, and wherein the at least one artery supplies blood to the patient's kidney.

10. The method of claim 1, wherein the at least one lipid-related condition is a cardiac condition, and wherein the at least one artery supplies blood to the patient's heart.

11. The method of claim 1, wherein the predetermined threshold value for the plaque burden is 4%.

12. The method of claim 1, wherein if, based on said determining, the plaque is the fibrous plaque, performing a fractional flow reserve assessment on the patient.

13. The method of claim 12, further comprising determining a percent of blockage due to lipid content.

14. The method of claim 13, wherein if the fractional flow reserve of the patient is equal to or above 80% and the percent of blockage due to lipid content is in a range of 20% to 30%, administering the delipidation treatment protocol to the patient.

15. The method of claim 14, wherein if the fractional flow reserve of the patient is below 80% or the percent of blockage due to lipid content is below 20% or above 30%, not administering the delipidation treatment protocol to the patient.

16. The method of claim 1, wherein if the plaque burden is less than 4%, not administering the delipidation treatment protocol to the patient.

17. The method of claim 1, wherein the at least one lipid-related condition is homozygous familial hypercholesterolemia, heterozygous familial hypercholesterolemia, Ischemic stroke, coronary artery disease, acute coronary syndrome, or peripheral arterial disease.

18. The method of claim 1, wherein, after administering to the patient the high-density lipoprotein composition, the patient experiences a reduction of plasma burden in the at least one artery that supplies blood to the patient's kidney.

19. The method of claim 1, further comprising:
determining, if the plaque is a fibrous plaque, a fractional flow reserve;
determining a third treatment protocol if the fractional flow reserve is less than or equal to 79%; and
determining a fourth treatment protocol if the fractional flow reserve is greater than 80%.

20. The method of claim 19, wherein the third treatment protocol comprises embedding a stent in the patient, and wherein the fourth treatment protocol comprises administering a medical therapy other than embedding a stent in the patient or administering the delipidation protocol to the patient.
